(19) United States
(12) Patent Application Publication
(10) Pub. No.: US 2004/0077529 A1
(43) Pub. Date: Apr. 22, 2004

(54) Urotensin II receptor agents

(76) Inventors: Per-Fredrik Lehmann, Saro (SE); Ingrid Kristina Luthman, Goteborg (SE); May-Britt Kary, Vallensbaek (DK); Carl-Magnus Andersson, (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/458,796

(22) Filed: Jun. 9, 2003

Related U.S. Application Data
(60) Provisional application No. 60/387,975, filed on Jun. 10, 2002.

(51) Int. Cl. A61K 38/17; A61K 31/66; A61K 31/4706; A61K 31/382; A61K 31/353; C07K 14/705

(52) U.S. Cl. 514/7; 514/12; 530/350; 514/109; 514/312; 514/456; 514/434

(57) ABSTRACT

Disclosed are compounds of Formula I, or salts or prodrugs thereof, complexed with a human urotensin II receptor

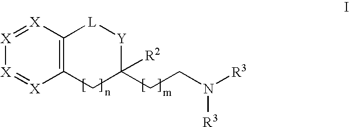

as defined herein. Also disclosed are compounds of Formula II, or salts or prodrugs thereof,

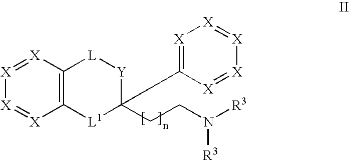

as defined herein. Also disclosed are methods of modulating the activity of a urotensin II receptor using a compound of Formula I, or a compound of Formula II, or salts or prodrugs thereof. In addition, methods of treating diseases related to the activity of urotensin II receptors are disclosed.

### RELATED APPLICATION

The present application claims priority to the U.S. Provisional Application Serial No. 60/387,975, filed Jun. 10, 2002, by Andersson, and entitled “UROTENSIN II RECEPTOR AGENTS,” the entire disclosure of which including any drawings, is hereby incorporated by reference herein.

### FIELD OF INVENTION

The present invention relates to non-endogenous modulators of the human urotensin II receptor. These human urotensin II receptor agonists are fused bicyclic systems di-substituted with an aromatic group and an aminoalkyl group.

### BACKGROUND OF THE INVENTION

Urotensin II is an, endogenous peptide agonist for a recently identified human G-protein coupled receptor. The human receptor is homologous to the rat orphan receptor GBP14.

Urotensin II is a cyclic neuropeptide found to be a potent vasoconstrictor in some systems and a vasodilator in others. The peptide is expressed in the motor neurons of the CNS, smooth muscle cells of the bladder and muscle cells of the heart. Its sequence is highly conserved among species, consisting of 11 amino acids in humans, 12 amino acids in fish, and 13 in frogs, with a fully conserved cyclic region from fish to humans.

The natural endogenous ligand, urotensin II, has been found to modulate the function of the urotensin II receptor. There is therefore a need in the art for non-endogenous ligands and modulators of the urotensin II receptor at least for use as medicaments.

Several responses to urotensin II have been observed in tissues and animals that may indicate physiological functions for this signalling molecule and its receptor and may indicate therapeutic uses of modulators of this system.

Human urotensin II has been reported as a potent spasmogen of primate airway smooth muscle and its contractile profile with pulmonary vascular tissue has showed that there were regional differences in its efficacy, with potent contractile activity on pulmonary arteries while having no effect in tissues distal from the atria (Br. J. Pharmacol., 131(1); 10-12).

Human urotensin II has been reported as an endothelium-dependent vasodilator in rat small arteries (Br. J. Pharmacol.; 130(8); 1865-1870). The human urotensin II peptide acts as a vasoconstrictor of rat and primate aorta, and induced a large increase in peripheral resistance in the circulation of primates along with a dramatic decrease in heart rate (Nature, 401; 282-286). In anesthetized rats, urotensin II peptide induced a decrease in blood pressure (General and Comparative Endocrinology 64; 435-439, Neuroendocrinol Lett. 14(5); 357-363). These results suggest that modulators of urotensin II and its receptor may alter cardiovascular function, particularly heart rate, cardiac output, peripheral resistance and arterial pressure.

Indications are that the physiological role of urotensin II in mammals is strongly tissue dependent. The mRNA for the human urotensin II receptor is widely expressed in human tissue and is most abundant in heart and pancreas. The cardiovascular tissue of the left atrium and ventricle of the heart, and arterial tissue such as in the aorta are especially rich in expression of the urotensin II receptor. Moreover, the receptor is also distributed within the smooth muscle cells of the bladder, coronary arteries, and the aorta, the endothelial cells of the coronary artery and umbilical vein, and the motor neurons of the spinal cord. The distribution of the pro-pre-urotensin II mRNA in the human central nervous system is restricted primarily to the medulla oblongata of the brain and the spinal cord with the urotensin II-like immunoreactivity localized to motor neurons of the ventral horn. The distribution of the pro-pre-urotensin II mRNA in peripheral tissue is primarily restricted to the adrenal glands, the kidneys and the spleen.

The physiological role that GPR-14 (the urotensin II receptor) serves in the mammalian central nervous system is currently unknown. Important insights into the possible physiological effects mediated by this G-protein coupled receptor can be gained from an understanding of which cells in brain express this gene. Recently, the pattern of expression of the mRNA that encodes this receptor was reported (1). The GPR-14 gene is expressed in a discrete, extremely limited distribution within the mammalian central nervous system. The only brain regions which express this mRNA are the pedunculopontine tegmental nucleus (PPT), and the lateral dorsal tegmental nucleus (LDTG). These brain stem nuclei are the source of the ascending acetylcholine projection neurons in mammals, and as such are quite well studied, and have had a number of important physiological roles assigned to them. The expression of this receptor gene in just these cholinergic neurons provides for a novel mechanism by which these cell groups can be selectively modulated by small organic compounds targeted to GPR-14.

Isocoumarins and ischromans substituted at the 3-position with a tertiary aminoethyl moiety and their preparation were disclosed in 1972 by Sandoz-Wander (U.S. Pat. No. 3,880,885). FR 72 11734 (Sandoz) discloses the isocoumarins 3-(2-dimethylamino-ethyl)-3,4-dihydro-3-phenyl-isocoumarin, 3-(2-dimethylamino-(1-methyl-ethyl))-3,4-dihydro-3-phenyl-isocoumarin and 3-(2-dimethylamino-ethyl)-3,4-dihydro-3-(p-methoxy-phenyl)-isocoumarin as diuretics by oral administration of the compounds to rats. FR 72.11734 further discloses the anti-hypertensive activity of 3-(2-dimethylamino-ethyl)-3,4-dihydro-3-phenyl-isocoumarin, 3-(2-dimethylamino-ethyl)-3,4-dihydro-3-(p-methoxy-phenyl)-isocoumarin and 3-(2-dimethylamino-ethyl)-3-(phenyl)-isochroman upon intravenal administration of the substances to dogs. The 3-(2-dimethylamino-(1-methyl-ethyl))-3,4-dihydro-3-phenyl-isocoumarin was found to be ineffective as an antihypertensive upon oral administration to rats. FR 72.11734 claims medicaments having diuretic and hypotensive/antihypertensive action comprising a compound selected from a list of isocoumarins and isochromans and inert excipients. 100121 More recently, 1-oxo-tetrahydronaphthalenes were described (U.S. Pat. No. 4,564,641) as having anti-depressant activity. Therapeutic compositions comprising a 1-oxo-tetrahydronaphthalene and pharmaceutical excipients as well as methods for treating mental disorders using a 1-oxo-tetrahydronaphthalene were claimed.

The art is silent in terms of intermediates involved in isocoumarin and isochroman physiological activity. Their mechanism of action is completely unknown. They have not been used as drugs targeted to act on a particular cellular process or molecular target involved in a disease-state. In order to understand and treat a disease-state, there is the need for agents targeted to specific processes and specific genetic targets involved in the development or manifestation of a disease or disorder.

### SUMMARY OF THE INVENTION

Disclosed are compounds of Formula I, or salts or prodrugs thereof, complexed with a human urotensin II receptor

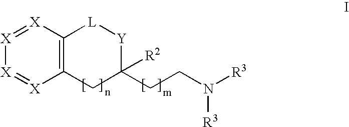

as defined herein.

Also disclosed are compounds of Formula II, or salts or prodrugs thereof,

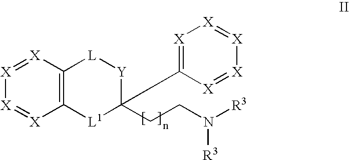

as defined herein.

Also disclosed are methods of modulating the activity of a urotensin II receptor using a compound of Formula I, or a compound of Formula II, or salts or prodrugs thereof. In addition, methods of treating diseases related to the activity of urotensin II receptors are disclosed.

### BRIEF DESCRIPTION OF THE DRAWINGS

FIG. 1 is a graph comparing the agonist activity of the compound of Formula III ( ) on the urotensin II receptor versus that of human urotensin II peptide (control) (−).

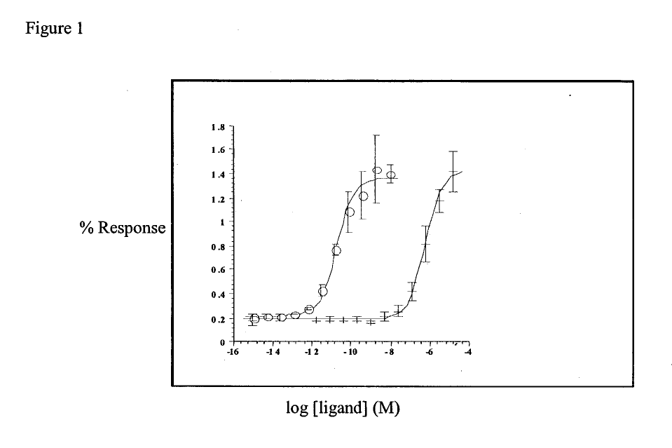

FIG. 2 is a graph showing the comparison of the effect on distance traveled between the compound of Formula III and two controls.

FIG. 3 is a graph showing the effect of the administration of the compound of Formula III on distance traveled.

FIG. 4 is a graph showing the effect of the administration of the compound of Formula III on vertical movements.

### DETAILED DESCRIPTION OF THE PREFERRED EMBODIMENTS

The present investigators have identified a class of non-endogenous, low molecular weight non-peptide organic compounds that act as specific modulators of the urotensin II receptor. Quite remarkably, the class of compounds, which produce a specific biological response through the urotensin II, receptor comprise fused bicyclic systems, such as isochromans and isocoumarins, di-substituted at the 3-position by an aromatic and an aminoalkyl.

Aspects of the present invention relate to a compound of Formula I or Formula II, as defined herein, or salts or prodrugs thereof. The compounds may appear as mixtures of isomers or as separated and purified isomers. Other aspects of the present invention relate to a complex between a human urotensin II receptor and a compound of the invention and to a method of preparing a complex between compound of the invention and human urotensin II receptor comprising combining said compound in an effective concentration with human urotensin II receptor.

The present inventors have demonstrated for the first time that compounds of the invention, namely compounds of Formula I, II, III, III-i, and III-ii, as defined herein, to be potent modulators of the human urotension II receptor. Correspondingly, a further aspect of the invention relates to a use of compound of Formula I, II, III, III-i, or III-ii, salts thereof, or compositions comprising said compounds, for the preparation of a medicament for the treatment of diseases and disorders for which activation or modulation of the urotensin II receptor produces a physiologically beneficial response in said disease or disorder. The diseases and disorders are selected from the group consisting of those associated with CNS function, such as Parkinson's Disease, Alzheimer's Disease, amylotrophic lateral sclerosis, muscular dystrophy, childhood spinal muscular atrophy, progressive spinal muscular atrophy and progressive bulbar palsy; OPCA; ADHD; schizophrenia; sleep disorders such as insomnia; and autonomic dysfunctions such as Shy Drager syndrome. Alternatively, the diseases or disorders are selected from the group consisting of cardiovascular disorders such as hypertension; hypotensive states related to shock, sepsis, major surgery and congestive heart failure.

As stated, a variety of disease states have been suggested to be associated with either an altered functioning of the urotensin II receptor or to an imbalance of urotensin II. For example, alteration of urotensin II and signalling through its cognate receptor may be associated with, amongst other disease-states, both hypertension and hypotension. Accordingly, a further aspect of the invention relates to method of altering the vascular pressure in a mammal, comprising constricting or dilating vascular tissue in said mammal, said constricting or dilating being performed by the activation of urotensin receptor signalling, said activation being performed by the administration of an effective amount of a compound of Formula I. Similarly, the invention relates to methods of altering the heart rate in a mammal, comprising the modulation of urotensin receptor signalling, said modulation being performed by the administration of an effective amount compound of Formula I.

Moreover, a method of treating diseases or disorders in a mammal, said diseases or disorders being associated with an altered urotensin II receptor activity or imbalance in urotensin II level or activity compared to urotensin receptor activity or urotensin II levels or activity in a mammal not having said disease or disorder, comprising administering an effective amount of a compound of Formula I is within the scope of the present invention. Accordingly, the present invention further relates to a method of treating diseases for which modulation of the urotensin II receptor produces a physiologically beneficial response in said disease, such as those associated with CNS function and cardiovascular diseases.

Remarkably, the present investigators have found that, upon administration of compounds of Formula I, the locomotor activity of the animal is altered. Accordingly, the invention further relates to a method of altering the locomotor activity of a mammal, comprising administering to said mammal an effective amount of a compound of Formula I.

This alteration of locomotor function may indicate a CNS-mediated response of a compound of Formula I and CNS mediated function of the urotensin II receptor that suggests application in CNS therapeutic areas. Thus, a further aspect of the invention relates to the treatment of diseases and disorders associated with CNS function. Given, the distribution of the urotensin II receptor within cardiovascular tissue, a further aspect of the invention relates to the treatment of cardiovascular disorders.

Thus, in a first aspect, the present invention relates to a compound of Formula I, or salts or prodrugs thereof, complexed with a human urotensin II receptor,

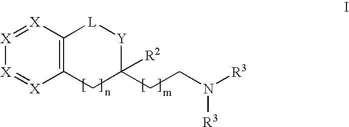

wherein

X is selected from the group consisting of CR¹ and N;

wherein each R¹ is independently and optionally selected from the group consisting of hydrogen, hydroxy, amino, halogen, optionally substituted aryl, optionally substituted heteroaryl, optionally substituted C₃₋₈-cycloalkyl, optionally substituted C₃₋₈ heterocyclyl, optionally substituted C(O)—R, optionally substituted C₁₋₆-alkyl, optionally substituted C₁₋₆-alkoxy, optionally substituted C₂₋₈-alkenyl and optionally substituted C₂₋₈-alkynyl;

R² is selected from the group consisting of optionally substituted aryl and optionally substituted heteroaryl;

each R³ is independently and optionally selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;

L is selected from the group consisting of CRR′, C(O), N(R³), S(O), S(O)₂, O, S, P, and P(O);

Y is absent or selected from the group consisting of CRR′, N—R³, O, S, and P;

m is an integer in the range from 0 to 5, such as 0, 1, 2, 3, 4, or 5;

n is an integer in the range from 0 to 3, such as 0, 1, 2, or 3; and

R and R′ are independently selected from the group consisting of hydrogen, hydroxy, amino, halogen, optionally substituted aryl, optionally substituted CI₆-alkyl, optionally substituted C₁₋₆-alkoxy, optionally substituted C₂₋₈-alkenyl and, optionally substituted C₂₋₈-alkynyl optionally substituted heteroaryl, optionally substituted C₃₋₈-cycloalkyl, and optionally substituted C₃₋₈heterocyclyl.

The terms “complexed with” or “complex between” are intended to mean a non-covalent bonding between a first and a second body induced by, for instance, electronic interactions, hydrophobic interactions, steric interactions, van der Waals forces and/or hydrogen bonding. Specifically, herein, it is intended to mean a non-covalent bonding interaction between a compound of Formula I, or a moiety or moieties thereof, and a site on the human urotensin II receptor, such as a binding site or an allosteric site.

In the present context, the term “alkyl” and “C₁₋₆-alkyl” are intended to mean a linear or branched saturated hydrocarbon chain wherein the longest chain has from one to six carbon atoms, such as methyl, ethyl, n-propyl, isopropyl, n-butyl, isobutyl, sec-butyl, tert-butyl, pentyl, isopentyl, neopentyl, and hexyl. An alkyl chain may be optionally substituted such as to form a C₁₋₆-alkyl(aryl), C₁₋₆-alkyl(heteroaryl), C₁₋₆-alkyl(heterocyclyl) or C₁₋₆-alkyl(C₃₋₈-cycloalkyl).

The term “C₁₋₆-alkyl(aryl)” is intended to mean a C₁₋₆-alkyl group substituted with an aryl group, each as defined herein. The aryl groups of C₁₋₆-alkyl(aryl) may be substituted or unsubstituted. Some examples include benzyl, substituted benzyl, 2-phenylethyl, 3-phenylpropyl, and naphthylalkyl.

In the present context the term “aryl” is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl. A preferred aryl group is phenyl. The term “aryl” relates to aromatic, preferably benzenoid groups connected via one of the ring-forming carbon atoms, and optionally carrying one or more substituents selected from halo, hydroxy, amino, cyano, nitro, alkylamido, acyl, C₁₋₆ alkoxy, C₁₋₆ alkyl, C₁₋₆ hydroxyalkyl, C₁₋₆ aminoalkyl, C₁₋₆ alkylamino, alkylsulfenyl, alkylsulfinyl, alkylsulfonyl, sulfamoyl, or trifluoromethyl. As stated, preferred aryl groups are phenyl, and, most suitably, substituted phenyl groups, carrying one or two, same or different, of the substituents listed above. A preferred pattern of substitution may be para and/or meta. Some examples of aryl groups include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 3-cyanophenyl, 4-cyanophenyl, dimethylphenyl, naphthyl, hydroxynaphthyl, hydroxymethylphenyl, trifluoromethylphenyl, alkoxyphenyl.

The term “C₁₋₆-alkyl(heteroaryl)” is intended to mean a C₁₋₆-alkyl group substituted with an heteroaryl group, each as defined herein. The heteroaryl groups of C₁₋₆-alkyl(heteroaryl) may be substituted or unsubstituted.

In the present context, the term “heteroaryl” is intended to mean a heterocyclic aromatic group where one or more carbon atoms in an aromatic ring have been replaced with one or more heteroatoms selected from the group comprising nitrogen, sulphur, phosphorous and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one C₃₋₈-cycloalkyl ring share at least one chemical bond.

The term “heteroaryl” is understood to relate to aromatic, C₂₋₆ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, preferably connected via one of the ring-forming carbon atoms. Heteroaryl groups may carry one or more substituents, selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. Preferred heteroaryl groups are five- and six-membered aromatic heterocyclic systems carrying 0, 1, or 2 substituents, which may be the same as or different from one another, selected from the list above. Some examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quionoline, isoquinoline, pyridazine, pyrimidine, purine and pyrazine, which are all preferred, as well as furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline. The most preferred substituents are halo, hydroxy, cyano, O—C₁₋₆-alkyl, C₁₋₆-alkyl, hydroxy-C₁₋₆-alkyl, amino-C₁₋₆-alkyl.

The term “C₁₋₆-alkyl(heterocyclyl)” is intended to mean a C₁₋₆-alkyl group substituted with a heterocyclyl group, each as defined herein. The heterocyclyl groups of C₁₋₆-alkyl(heterocyclyl) may be substituted or unsubstituted.

The term “heterocyclyl” is intended to mean three-, four-, five-, six-, seven-, and eight-membered rings wherein carbon atoms together with from 1 to 3 heteroatoms constitute said ring. A heterocyclyl may optionally contain one or more unsaturated bonds situated in such a way, however, that an aromatic π-electron system does not arise. The heteroatoms may be independently selected from oxygen, sulphur, and nitrogen. A heterocyclyl may further contain one or more carbonyl or thiocarbonyl functionalities, so as to make the definition include oxo-systems and thio-systems such as lactams, lactones, cyclic imides, cyclic thioimides, cyclic carbamates, and the like. Heterocyclyl rings may optionally also be fused to aryl rings, such that the definition includes bicyclic structures. Preferred such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” are the heterocycles tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, thiazoline, thiazolidine, or 1,3-oxathiolane. Binding to the heterocycle may be at the position of a heteroatom or via a carbon atom of the heterocycle, or, for benzo-fused derivatives, via a carbon of the benzenoid ring.

The term “C₁₋₆-alkyl(C₃₋₈-cycloalkyl)” is intended to mean a C₁₋₆-alkyl group substituted with a C₃₋₈-cycloalkyl group, each as defined herein. The C₃₋₈-cycloalkyl groups of C₁₋₆-alkyl(C₃₋₈-cycloalkyl) may be substituted or unsubstituted.

In the present context, the term “C₃₋₈-cycloalkyl” is intended to cover three-, four-, five-, six-, seven-, and eight-membered rings comprising carbon atoms only. A C₃₋₈-cycloalkyl may optionally contain one or more unsaturated bonds situated in such a way, however, that an aromatic π-electron system does not arise.

Some examples of “C₃₋₈-cycloalkyl” are the carbocycles cyclopropane, cyclobutane, cyclopentane, cyclopentene, cyclopentadiene, cyclohexane, cyclohexene, 1,3-cyclohexadiene, 1,4-cyclohexadiene, cycloheptane, or cycloheptene.

In the present context, the term “C₂₋₈-alkenyl” is intended to mean a linear or branched hydrocarbon group having from two to eight carbon atoms and containing one or more double bonds. Some examples of C₂₋₈-alkenyl groups include allyl, homo-allyl, vinyl, crotyl, butenyl, pentenyl, hexenyl, heptenyl and octenyl. Some examples of C₂₋₈-alkenyl groups with more than one double bond include butadienyl, pentadienyl, hexadienyl, heptadienyl, heptatrienyl and octatrienyl groups as well as branched forms of these. The position of the unsaturation (the double bond) may be at any position along the carbon chain.

In the present context the term “C₂₋₈-alkynyl” is intended to mean a linear or branched hydrocarbon group containing from two to eight carbon atoms and containing one or more triple bonds. Some examples of C₂₋₈-alkynyl groups include ethynyl, propynyl, butynyl, pentynyl, hexynyl, heptynyl and octynyl groups as well as branched forms of these. The position of unsaturation (the triple bond) may be at any position along the carbon chain. More than one bond may be unsaturated such that the “C₂₋₈-alkynyl” is a di-yne or enedi-yne as is known to the person skilled in the art.

When used herein, the term “O—C₁₋₆-alkyl” is intended to mean C₁₋₆-alkyloxy, or alkoxy, such as methoxy, ethoxy, n-propoxy, isopropoxy, n-butoxy, isobutoxy, sec-butoxy, tert-butoxy, pentyloxy, isopentyloxy, neopentyloxy and hexyloxy

The term “halogen” includes fluorine, chlorine, bromine and iodine.

In the context of the present, for example, in connection with the terms “C₁₋₆-alkyl”, “aryl”, “heteroaryl”, “heterocyclyl”, “C₃₋₈-cycloalkyl”, “heterocyclyl(C₁₋₆-alkyl)”, “(cycloalkyl)alkyl”, “O—C₁₋₆-alkyl”, “C₂₋₈-alkenyl”, and “C₂₋₈-alkynyl”, the term “optionally substituted” is intended to mean that the group in question may be substituted one or several times, such as 1 to 5 times, preferably 1 to 3 times, most preferably 1 to 2 times, with one or more groups selected from C₁₋₆-alkyl, C₁₋₆-alkoxy, oxo (which may be represented in the tautomeric enol form), carboxyl, amino, hydroxy (which when present in an enol system may be represented in the tautomeric keto form), nitro, alkylsulfonyl, alkylsulfenyl, alkylsulfinyl, C₁₋₆-alkoxycarbonyl, C₁₋₆-alkylcarbonyl, formyl, amino, mono-and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C-₁₋₆-alkylsulphonyloxy, dihalogen-C₁₋₆-alkyl, trihalogen-C₁₋₆-alkyl, and halo. In general, the above substituents may be susceptible to further optional substitution.

The term “salts” is intended to mean pharmaceutically acceptable acid addition salts obtainable by treating the base form of a functional group, such as an amine, with appropriate acids such as inorganic acids, for example hydrohalic acids; typically hydrochloric, hydrobromic, hydrofluoric, or hydroiodic acid; sulfuric acid; nitric acid; phosphoric acid and the like; or organic acids, for example acetic, propionic, hydroacetic, 2-hydroxypropanoic acid, 2-oxopropanoic acid, ethandioic, propanedioic, butanedioic, (Z)-2-butenedioic, (E)-butenedioic, 2-hydroxybutanedioic, 2,3-dihydroxybutanedioic, 2-hydroxy-1,2,3-propanetricarboxylic, methanesulfonic, ethanesulfonic, benzenesulfonic, 4-methylbenzenesulfonic acid, cyclohexanesulfamic, 2-hydoxybenzoic, 4-amino-2-hydroxybenzoic, and other acids known to the skilled practitioner.

The present invention includes within its scope prodrugs of the compounds of this invention. In general, such prodrugs are inactive derivatives of the compounds of this invention that are readily convertible in vivo into the required compound. Conventional procedures for the selection and preparation of suitable prodrug derivatives are described, for example, in Design of Prodrugs, (ed. H. Bundgaard, Elsevier, 1985). Metabolites of these compounds include active species that are produced upon introduction of compounds of this invention into the biological milieu.

It should also be understood that salts of compounds of Formula I, II or III other than ammonium salts are anticipated, including for instance hydrates and solvent addition forms. Moreover, base addition salts are anticipated, including alkali metals, such as sodium and potassium, alkali earth metals, such as calcium and magnesium, and organic addition salts such as quaternary ammonium cations.

For purposes of chemical logic, it should be noted that in a compound of Formula I,

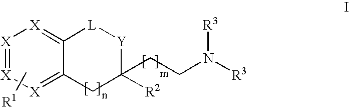

R¹ is present no more than 4 times, such as from 0 to 4 times, such as 0, 1, 2, 3, or 4 times.

The compound of Formula I is a fused bicyclic benzenoid system which is di-substituted by an aromatic group and an aminoalkyl group. The fused bicycle is a system wherein an aromatic ring shares two carbons with a heterocycle or carbocycle. The heterocycle or carbocycle may be a 4, 5, 6, 7, or 8-membered ring. In the embodiment where the heterocycle or carbocycle is a 4-membered ring, Y is absent and n is 0. The heterocycle or carbocycle may be a 5-membered ring such that one of the carbons shared by both rings is bonded directly to the position of di-substitution, i.e. n is 0. Alternatively, the heterocycle or carbocycle may be a 5-membered ring such that L is bonded directly to the position of bis-substitution, i.e. Y is absent.

The integer n determines the size of the heterocycle or carbocycle and may be in the range of 0 to 3, such as 0, 1, 2, or 3. In some embodiments, n is 1.

The size of the carbocycle or heterocycle is also determined by the presence or absence of Y. Y may be absent or selected from the group consisting of CRR′, N—R³, oxygen, sulfur, and phosphorous. In certain embodiments, Y is present and is selected from the group consisting of carbon, nitrogen, and oxygen. In some embodiments, Y is oxygen.

In certain embodiments where Y is CRR′, the carbocycle or heterocyle of the benzenoid system may be substituted adjacent to the bis-substituted position, given R or R′ may be independently selected from the group consisting aryl, heteroaryl, C₃₋₈-cycloalkyl, heterocyclyl, C₁₋₆-alkyl, C₁₋₆-alkoxy, C₂₋₈-alkenyl, C₂₋₈-alkynyl, C₁₋₆-alkylsulfonyl, and arylsulfonyl, each of which being optionally substituted; hydrogen and halogen. In other embodiments wherein neither R or R′ is hydrogen, the carbocycle or heterocyle may thus be bis-substituted adjacent to the position of bis-substitution. In some embodiments where Y is CRR′, either R or R′ may be hydrogen and the other may be selected from the group consisting aryl, heteroaryl, C₃₋₈-cycloalkyl, heterocyclyl, C₁₋₆-alkyl, C₁₋₆-alkoxy, C₂₋₈-alkenyl, C₂₋₈-alkynyl, C₁₋₆-alkylsulfonyl, and arylsulfonyl, each of which being optionally substituted, and halogen. In further embodiments, Y may be selected from the group consisting of CRR′, N—R³ and oxygen. In some embodiments wherein Y is CRR′, both R and R′ may be hydrogen.

In some embodiments, L may be selected from the group consisting of CRR′, C(O), N—R³, S(O), S(O)₂, oxygen, sulfur, phosphorous, and P(O). In certain embodiments, L may be selected from the group consisting of CRR′, C(O), and P(O), most preferably from CRR′ and C(O). In certain other embodiments, L may be C(O) and Y may be oxygen such that the heterocycle formed is a lactone. Alternatively, L may be CRR and Y may be oxygen such that the heterocycle formed is an optionally substituted cyclic ether. In other embodiments, L may be C(O) and Y may be nitrogen, such that the heterocycle formed is a lactam. In further embodiments, L or Y may be selected such that the heterocycle formed is an alkaloid.

In still other embodiments of the invention, the carbocycle or heterocycle may be a cyclic ketone, a cyclic ether, a lactone, a lactam, a lactam, or a cyclic amide.

As stated, the fused bicycle may be a benzenoid system wherein an aromatic ring shares two carbons with a heterocycle or carbocycle. Alternatively, the fused bicycle may comprise a heteroaromatic ring sharing two carbons with a heterocycle or carbocycle, that is to say that at least one X may be nitrogen. There may, however, be more than one heteroatom in the heteroaromatic ring. In some embodiments, where there is one heteroatom in the aromatic ring, the heteroatom may be at any position within the ring. In certain embodiments, at most one X is nitrogen, and the remainder are CR¹.

In certain embodiments of the invention, the fused bicyclic benzenoid system may comprise an aryl, i.e. each X may be carbon.

In further embodiments, X may be carbon, Y may not be absent, and n may be 1. In other embodiments, Y may be oxygen and L may be selected from CRR and C(O).

In some embodiments, the aryl or heteroaryl of the fused bicyclic ring system may be optionally substituted from 0 to 4 times with R¹, such as 1, 2, 3, or 4 times. R¹ may be a monoradical or biradical selected from the group consisting of aryl, heteroaryl, C₃₋₈-cycloalkyl, C₃₋8 heterocyclyl, C(O)—R, C₁₋₆-alkyl, C₁₋₆-alkoxy, C₂₋₈-alkenyl and C₂₋₈-alkynyl, each of which may be optionally substituted, hydrogen, hydroxyl, and halogen. Moreover, R¹ may be a biradical such that the aryl or heteroaryl of the fused bicyclic ring system may be substituted at two positions with the biradical R¹ so as to form a further fused ring system, i.e. that R¹ forms a ring system by sharing two carbons with the aryl or heteroaryl. Alternatively, R¹ may be a biradical so as to form a spiro ring system with the benzenoid ring system.

In certain embodiments, R¹ may be a monoradical and may be selected from the group consisting of C₁₋₆-alkyl, C₁₋₆-alkoxy, hydrogen, hydroxy, and halogen.

In further embodiments, L may be selected from the group consisting of CRR′ and C(O), Y may be oxygen, and X may be carbon.

Some examples of compounds of Formula I are

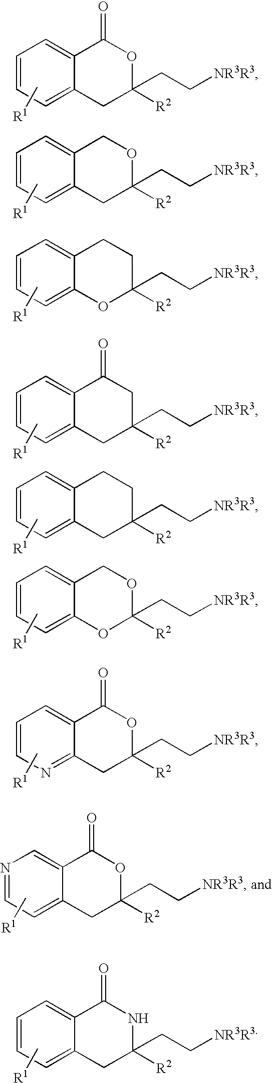

Depending on the nature of R³, the aminoalkyl moiety may be primary amine, a secondary amine, a tertiary amine, an amide or a quaternary ammonium salt. R³ may be selected from the group consisting of aryl, heteroaryl, C₃₋₈-cycloalkyl, C₃₋₈ heterocyclyl, C₁₋₆-alkyl, C₂₋₈-alkenyl and C₂₋₈-alkynyl, each of which may be optionally substituted, hydrogen, and C(O)R. In some embodiments, R³ may be selected from the group consisting of aryl, C₁₋₆-alkyl, and C₃₋₈-cycloalkyl, each of which may be optionally substituted. Otherwise stated, the aminoalkyl moiety may be a tertiary amine.

In some embodiments, R³ may be selected from the group consisting of optionally substituted aryl, optionally substituted C₁₋₆-alkyl, and optionally substituted C₃₋₈-cycloalkyl. In further embodiments, R³ may be an optionally substituted C₁₋₆-alkyl(aryl). In other embodiments, both R³ groups may be the same, and may be a C₁₋₆-alkyl. In yet other embodiments, R³ may be selected from a C₁-alkyl, a C₂-alkyl and a C₃-alkyl.

In some embodiments, two R³s and the nitrogen atom to which they are attached form a heterocyclyl or heteroaryl ring. When two substituents taken together along with the nitrogen atom to which they are attached form a heteroaryl or heterocyclyl ring, it is meant that the following structure:

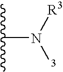

is, for example, representative of the following structures:

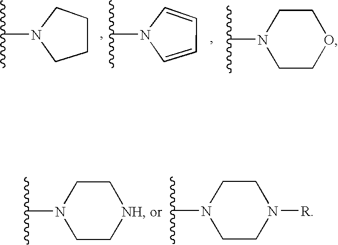

The aminoalkyl may be tethered to the fused bicylic system by a saturated carbon chain. The value of m determines the length of the aliphatic chain. The value of m may be selected from the range of 0 to 5, such as 0, 1, 2, 3, 4, or 5. In some embodiments, the tether may be the length of an ethylamine. Accordingly, in these embodiments m is 1.

In some embodiments, the fused bicyclic benzenoid system may be di-substituted with the aminoalkyl and R². The substituent R² may be selected from the group comprising aryl and heteroaryl, each of which may be optionally substituted. In further embodiments, R² may be selected from the group comprising aryl and heteroaryl substituted 0 to 3 times, such as 0, 1, 2, or 3 times, or 0 to 2 times, or 1 or 2 times. In some embodiments, R² may be substituted in the para-position, while in other embodiments it may be substituted in the meta-position, and in still other embodiments, it may be substituted in meta- and para-positions, both.

In certain embodiments, R² may be selected from the group comprising aryl and heteroaryl, substituted 1 to 2 times with a substituent, in the para-position in some embodiments, in the meta-position in other embodiments, or in meta- and para-positions in still other embodiments. The substituent may be selected from the group consisting of aryl, heteroaryl, C₃₋₈-cycloalkyl, C₃₋₈-heterocyclyl, C₁₋₆-alkyl, C₁₋₆-alkoxy, C₂₋₈-alkenyl and C₂₋₈-alkynyl, each of which may be optionally substituted, hydrogen, hydroxy, and halogen. In some embodiments, R² may be selected from the group comprising aryl and heteroaryl, substituted 1 to 2 times with hydroxy, halogen, C₁₋₆-alkoxy, C₃₋₈-heterocyclyl, aryl and heteroaryl.

In certain embodiments, R² may be selected from the group consisting of aryl and heteroaryl, substituted 0 to 3 times, such as 0, 1, 2, or 3 times, or 0, 1, or 2 times, or 1 or 2 times. In some embodiments, R² may be selected from the group consisting of aryl and heteroaryl, substituted 0 to 3 times, such as 1 or 2 times, in the para-position in some embodiments, in the meta-position in other embodiments, or in meta- and para-positions in still other embodiments. In certain embodiments, R² may be selected from the group consisting of aryl and heteroaryl, substituted 1 or 2 times with a substituent selected from the group consisting of aryl, heteroaryl, C₃₋₈-cycloalkyl, C₃₋₈ heterocyclyl, C₁₋₆-alkyl, C₁₋₆-alkoxy, C₂₋₈-alkenyl and C₂₋₈-alkynyl, each of which may be optionally substituted, hydrogen, hydroxy, and halogen, or substituted 1 to 2 times with hydroxy, halogen, optionally substituted C₁₋₆-alkoxy, C₃₋₈ heterocyclyl, aryl and heteroaryl.

Some examples of compounds of Formula I are

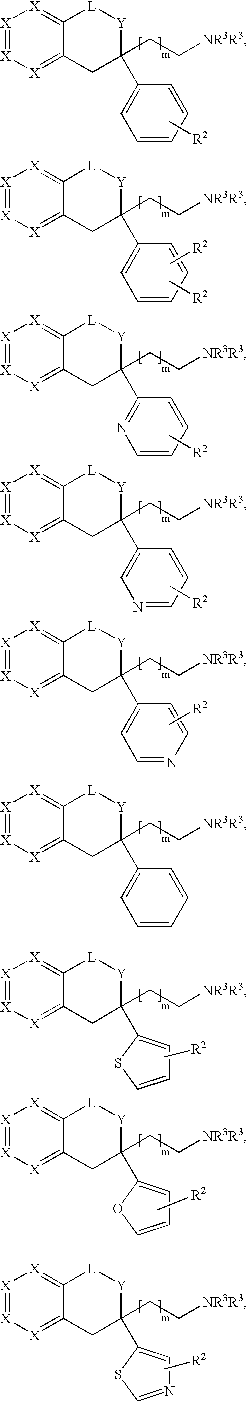

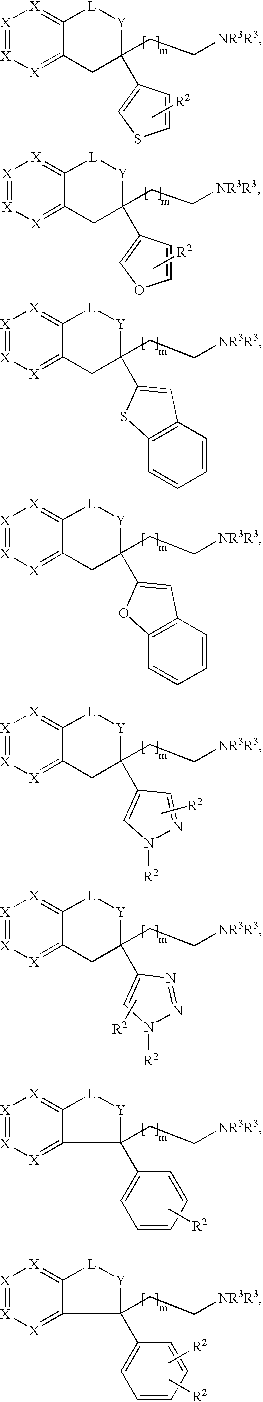

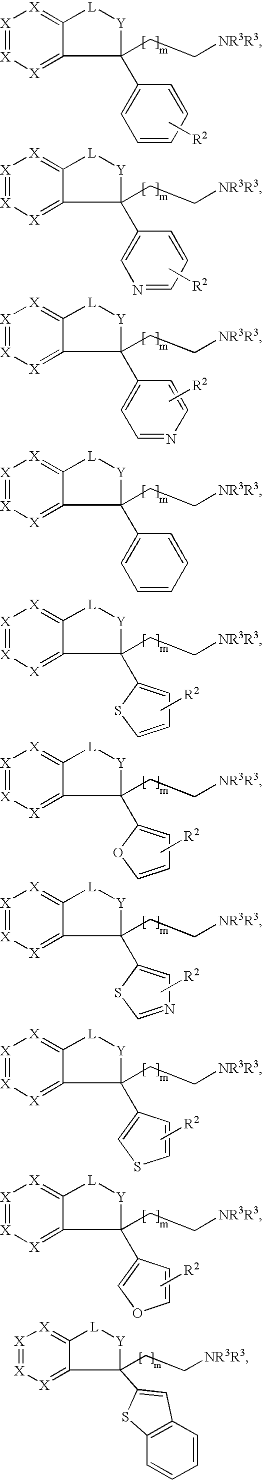

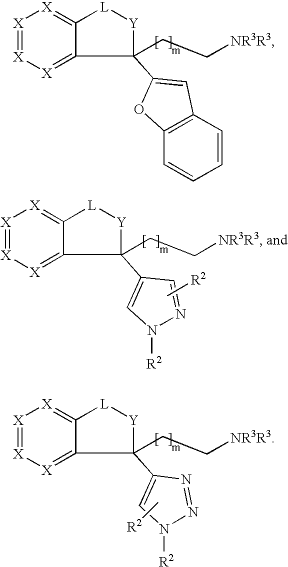

The compounds of Formula I have surprising activity as modulators of urotensin II receptor. Accordingly, one aspect of the present invention relates to a complex between a compound of Formula I and a human urotensin II receptor. Furthermore, not only have the present inventors identified the compounds of Formula I as modulators to the human urotensin II receptor, but also as agonists to said receptor. Accordingly, another aspect of the invention relates to a method of increasing the activity of the urotensin II receptor comprising providing a compound of Formula I to a system comprising said receptor. The increase in activity is measured by the increase in signalling as identified by Method 1, set forth in Example 1, below.

The complex may be in a partially or substantially purified form or may be in a complex mixture, solution or test system. The presence of complex is substantiated by the result of Method 1. Thus, another aspect of the invention relates to a complex comprising a urotensin II receptor and a compound that results in a positive performance evaluation according to Method 1. Thus, in another aspect of the invention, the compound of said complex is characterized in that it performs positively in the test conditions of Method 1 and is not urotensin. Preferably it is a compound of Formula I.

In another aspect, the present invention relates to a compound of Formula II, or salts, or prodrugs, or quaternary ammonium salts thereof,

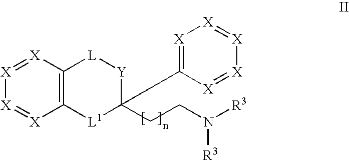

wherein

each of the four R¹ groups is independently selected from the group consisting of hydrogen, hydroxy, halogen, optionally substituted C₁₋₆-alkyl, optionally substituted aryl, optionally substituted heteroaryl, optionally substituted C₃₋₈-cycloalkyl, optionally substituted C₃₋₈ heterocyclyl, optionally substituted C(O)—R, optionally substituted C₁₋₆-alkoxy, optionally substituted C₂₋₈-alkenyl and optionally substituted C₂₋₈-alkynyl;

R³ is selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;

X is selected from the group consisting of CR² and N; wherein R² is independently selected from the group consisting of hydrogen, hydroxy, halogen, optionally substituted C₁₋₆-alkyl, optionally substituted aryl, optionally substituted heteroaryl, optionally substituted C₃₋₈-cycloalkyl, optionally substituted C₃₋₈ heterocyclyl, optionally substituted C(O)—R, optionally substituted C₁₋₆-alkoxy, optionally substituted C₂₋₈-alkenyl and optionally substituted C₂₋₈-alkynyl;

L and L¹ are independently selected from the group consisting of CRR′, C(O), C(S), N(O), N—R³, S(O), S(O)₂, oxygen, sulfur, phosphorous, and P(O), with the proviso that if L¹ is C(O), L is not CH₂;

Y is selected from the group consisting of CRR′, N—R³, oxygen, sulfur, and phosphorous;

n is an integer in the range from 0 to 5, such as 0, 1, 2, 3; 4 or 5; and

R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃₋₈ heterocyclyl.

As will be known to the person skilled in the art, R¹ may be present from 0-4 times, such as 0, 1, 2, 3, or 4 times and R² may be present from 0 to 5 times, such as 0, 1, 2, 3, 4, or 5 times. In some embodiments, compounds of Formula II may be such that when n is 1, L¹ is C(O), L is CH₂, X is carbon and none of the R³ groups are hydrogen, Y is not CH₂. Moreover, in other embodiments, when n is 1, L¹ is CH₂, X is carbon, L is CH₂ or C(O), and none of the R³ groups are hydrogen, Y is not oxygen.

In certain embodiments, the present invention relates to a compound of Formula II, where X may be selected form the group consisting of CR² and nitrogen. In some embodiments, at most one X is nitrogen, and the remainder are CR². In other embodiments, each X is carbon. In further embodiments, each X is carbon and 0 to 3 of the carbons are substituted such as 0, 1, 2, or 3 of the carbons are substituted with R². In still other embodiments, 1 to 3 of the carbons are substituted, while in other embodiments, 1 to 2 of the carbons are substituted. In some embodiments, the substitutions are in the para-position, while in other embodiments, the substitutions are in the meta-position, and in still other embodiments, the substitutions are in both meta- and para-positions.

In some embodiments, the substituent R² may be selected from the group comprising aryl, heteroaryl, C₃₋₈-cycloalkyl, C₃₋₈ heterocyclyl, C(O)—R, C₁₋₆-alkyl, C₁₋₆-alkoxy, C₂₋₈-alkenyl and C₂₋₈-alkynyl, each of which may be optionally substituted, hydrogen, hydroxy, and halogen. In other embodiments, R² may be an electron withdrawing group or may be selected from the group comprising aryl, heteroaryl, C₁₋₆-alkoxy, hydroxy, and halogen. In further embodiments of a compound of Formula II, R³ may be selected from the group consisting aryl, C₁₋₆-alkyl, and C₃₋₈-cycloalkyl.

In certain embodiments, one X is nitrogen, and the remainder are CR².

In connection to the amine of Formula II, in some embodiments R³ may be such that the amine is primary, such that both of the R³ groups are hydrogen; secondary such that one R³ group is hydrogen and the other is selected from aryl, heteroaryl, C₃₋₈-cycloalkyl, C₃₋₈ heterocyclyl, C₁₋₆-alkyl, C₂₋₈-alkenyl and C₂₋₈-alkynyl, each of which may be optionally substituted; or tertiary wherein each R³ is independently selected from aryl, heteroaryl, C₃₋₈-cycloalkyl, C₃₋₈ heterocyclyl, C₁₋₆-alkyl, C₂₋₈-alkenyl and C₂₋₈-alkynyl, each of which may be optionally substituted; or such that the nitrogen may be comprised within an amide, such that one R³ is C(O)—R and the other R³ is selected from the group consisting of aryl, heteroaryl, C₃₋₈-cycloalkyl, C₃₋₈ heterocyclyl, C₁₋₆-alkyl, C₂₋₈-alkenyl and C₂₋₈-alkynyl, each of which being optionally substituted, and hydrogen.

Still in connection with the alkylamine Formula II, in certain embodiments the length of the aliphatic chain may be such that n is 1.

Some examples of compounds of Formula II are

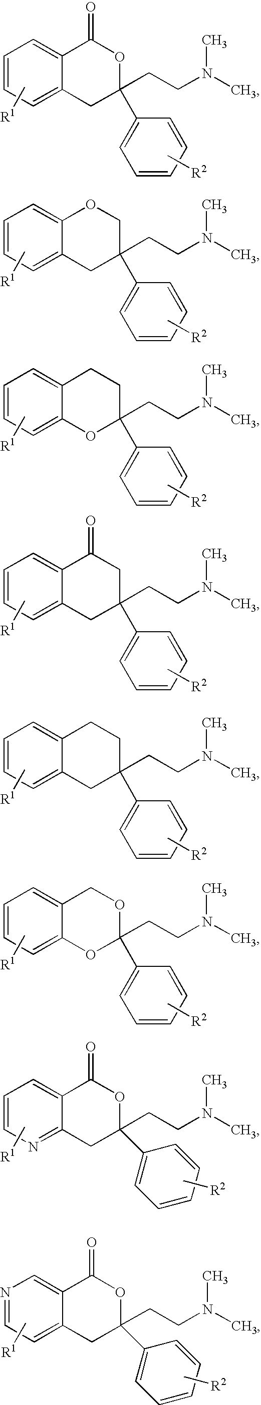

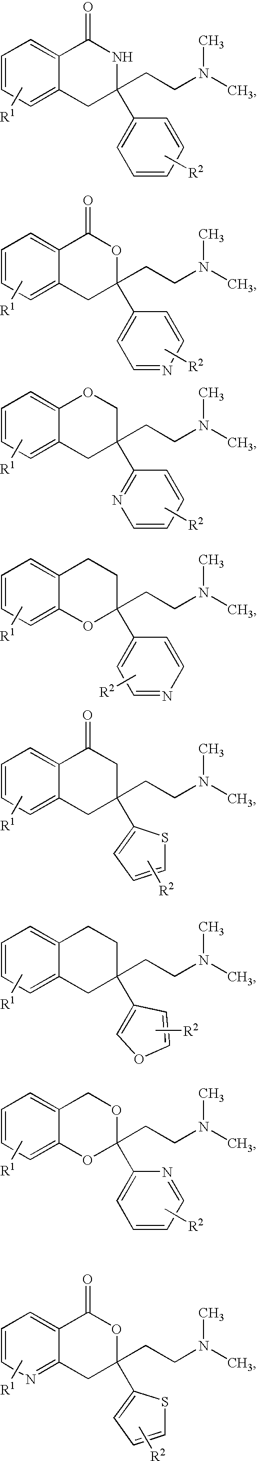

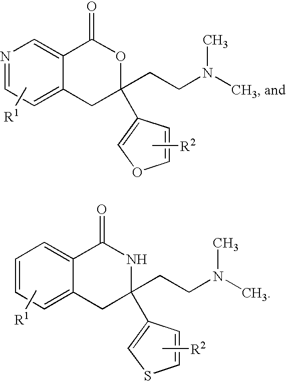

The compound of Formula II according to the present invention may be in an enantio-enriched form. As demonstrated in Example 3, below, the present inventors have separated enantiomeric forms of compounds of Formula II. As is known to the person skilled in the art, substituents present on compounds of Formula II may provide a further chiral center so as to lead to possible diastereomers of compounds of Formula II. A further aspect of the invention relates to diasteromeric mixtures of compounds of Formula II, enriched diasteromeric mixtures of compounds of Formula II, isolated diastereomers of compounds of Formula II, enantiomeric mixtures of compounds of Formula II, enriched enantiomeric mixtures of compounds of Formula II and isolated enantiomers of compounds of Formula II. The term “isolated” in connection to the diastereomers and enantiomers is intended to mean at least 90% purity, preferably at least 95% purity, more preferably at least 98% purity, and most preferably 99% purity.

Another aspect of the present invention relates to compounds of Formula II for use as a medicament. Compounds of Formula II may act as a medicament through the urotensin II receptor. It is also anticipated that compounds of Formula II will be useful as medicaments for ailments not necessarily associated with urotensin.

Thus, another aspect of the present invention relates to a method of treating a disease associated with the activity of the urotensin II receptor, comprising identifying individuals in need of such treatment and administering a compound of the present invention to said individuals. In another aspect, the present invention relates to a method of treating a disease associated with the activity of the urotensin II receptor, comprising identifying individuals in need of such treatment and contacting a compound of the present invention to said individuals.

The present inventors have surprisingly. separated compounds of Formula I into pure isomers and isomer enriched mixtures. A further aspect of the present invention relates to a compound of Formula I

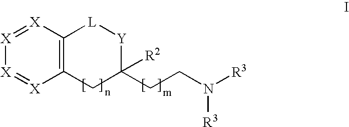

having the same absolute configuration as the compound of Formula III-i

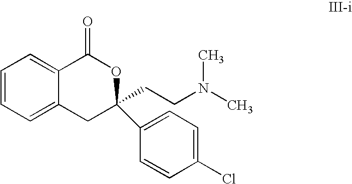

wherein X, R², R³, L, Y, n, and m are as defined herein, wherein said compound is essentially free of any of its enantiomers or diastereomers.

The other isomer of a compound of Formula I is also an aspect of the present invention, relating to a compound of Formula I

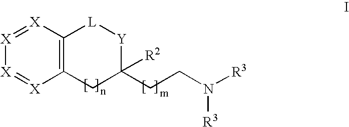

having the same absolute configuration as the compound of Formula III-ii

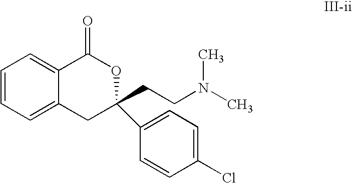

wherein X, R², R³, L, Y, n, and m are as defined herein, wherein said compound is essentially free of any of its enantiomers or diastereomers.

By “essentially free” it is meant that the stereoisomer of interest comprises at least 90%, or at least 95%, or at least 99%, of the compound of Formula I in the solution or in the solid mixture where it is found.

Similarly, another aspect of the present invention relates to a compound of Formula II having the same absolute configuration as the compound of Formula III-i. A further aspect of the invention relates to a compound of Formula II having the same absolute configuration as the compound of Formula III-ii. In certain embodiments, each of the compounds of Formula II having the absolute configuration as the compound of Formula III-i or III-ii is found essentially free of the other isomer.

The term “same absolute configuration” is intended to mean a compound being homochiral, here, with a compound of Formula III-i or III-ii. It must be noted that the compounds have the same handedness but may not have the same designator, as will be known to the person skilled in the art.

The present invention thus further relates to a compound of Formula I with an enantiomeric excess of more than 1% of the 1-R or 1-S enantiomer. The invention further relates to a compound of Formula II having an enantiomeric excess of more than 1% of the 1-R or 1-S enantiomer. As stated, the present inventors have separated isomeric mixtures of compounds of Formula I and II, typically such that enantiomeric excess is at least 50%, such as at least 60%, 70%, or 80%, preferably at least 90%, such as at least 95%, such as 96%, 97%, 98%, and 99%.

In another aspect, the present invention relates to a compound selected from the group consisting of:

3-(2-Dimethylaminoethyl)-3-phenyl-isochroman-1-one;

3-(2-Dimethylaminoethyl)-5-methyl-3-phenyl-isochroman-1-one;

3-(2-Dimethylaminoethyl)-7-methyl-3-phenyl-isochroman-1-one;

3-(2-Dimethylaminoethyl)-6-methyl-3-phenyl-isochroman-1-one;

3-(2-Dimethylaminoethyl)-5-methoxy-3-phenyl-isochroman-1-one;

3-(2-Dimethylaminoethyl)-5-fluoro-3-phenyl-isochroman-1-one;

3-(4-Chlorophenyl)-3-[2-(pyrrolidin-1-yl)-ethyl]-isochroman-1-one;

3-(4-Chlorophenyl)-3-(2-dimethylaminoethyl)-6-methyl-isochroman-1-one;

3-(4-Chlorophenyl)-3-[2-(piperidin-1-yl)-ethyl]-isochroman-1-one;

3-(4-Chlorophenyl)-3-(2-dimethylaminoethyl)-5-methoxy-isochroman-1-one;

3-(4-Chlorophenyl)-3-[2-(morpholin-1-yl)-ethyl]-isochroman-1-one;

3-(4-Chlorophenyl)-3-[2-(4-methyl-piperazin-1-yl)-ethyl]-isochroman-1-one;

3-(2-Dimethylaminoethyl)-3-(4-trifluoromethyl-phenyl)-isochroman-1-one;

3-(4-Chlorophenyl)-3-(2-dimethylaminoethyl)-7-methyl-isochroman-1-one;

3-(4-Chlorophenyl)-3-(2-dimethylaminoethyl)-5-methyl-isochroman-1-one;

3-(2-Dimethylaminoethyl)-3-(4-methyl-phenyl)-isochroman-1-one;

3-(2-Dimethylaminoethyl)-3-(4-methoxy-phenyl)-isochroman-1-one;

3-(2-Dimethylaminoethyl)-3-(3-methoxyphenyl)-isochroman-1-one;

3-(2-Dimethylaminoethyl)-3-(3-fluorophenyl)-isochroman-1-one;

3-(2-Dimethylaminoethyl)-3-(2-methoxyphenyl)-isochroman-1-one;

3-(2-Dimethylaminoethyl)-3-(4-phenoxyphenyl)-isochroman-1-one;

3-(2-Dimethylaminoethyl)-3-(2-chlorophenyl)-isochroman-1-one;

3-(4-Chlorophenyl)-3-(2-diethylaminoethyl)-isochroman-1-one;

3-(4-Chlorophenyl)-3-(2-dimethylaminoethyl)-4-methyl-isochroman-1-one;

3-(3-Chlorophenyl)-3-(2-dimethylaminoethyl)-isochroman-1-one;

3-(4-Chlorophenyl)-3-(2-dimethylaminoethyl)-8-methyl-isochroman-1-one;

3-(4-Chlorophenyl)-3-(3-dimethylaminopropyl)-isochroman-1-one;

3-(2-Dimethylaminoethyl)-3-(1-naphtyl)-isochroman-1-one;

3-(2-Dimethylaminoethyl)-3-(2-naphtyl)-isochroman-1-one; and

3-(2-Dimethylaminoethyl)-3-(2-thienyl)-isochroman-1-one.

In another embodiment, the present invention relates to an HCl salt of one of the above compounds.

In certain embodiments, the compound of Formula I or II is a compound of Formula III. A further aspect of the invention thus relates to a compound of Formula III,

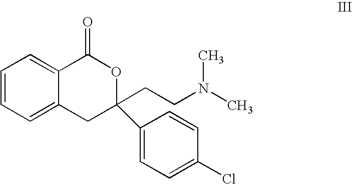

In a preferred embodiment of the invention relating to a compound of Formula III, the compound of Formula III preferably has an optical rotation. As can be seen from the Examples, the compounds of Formula III, when isolated so as to be enantiomerically enriched, has chirality and deflects plane polarized light. In a preferred embodiment, the compound of Formula III has an optical rotation selected from the group consisting of +50 to +59.5 and −50 to −59.5, most preferably such that the optical rotation is selected from the group consisting of +55 to +59.5 and −55 to −59.5.

In a suitable embodiment of a compound of Formula III, the optical rotation is selected from the group consisting of +56 to +59.5, such as +57 to +59.5, preferably +57.5 to +59.3; and −56 to −59.5, such as −57 to −59.5, preferably −57.5 to −59.3.

In a preferred embodiment of a compound of Formula III, the compound has an enantiomeric excess of at least 50%, such as at least 60%, 70%, or 80%, preferably at least 90%, such as at least 95%, such as 96%, 97%, 98%, and 99%. In a combination of preferred embodiments, the compound of Formula III has an enantiomeric excess of at least 50%, an optical rotation of −50 to −60; and has an elution time, under the liquid chromatographic conditions of Method A, of 12 to 16 minutes. Preferably the compound of Formula III has The compound has an enantiomeric excess is of at least 75%; and wherein the optical rotation of is −55 to −60.

In a preferred embodiment, the compound of Formula I has an enantiomeric excess of at least 50%, such as at least 60%, 70%, or 80%, preferably at least 90%, such as at least 95%, such as 96%, 97%, 98%, and 99%. %. In a combination of preferred embodiments, the compound of Formula III has an enantiomeric excess of at least 50%, an optical rotation of +50 to +60; and an elution time, under the liquid chromatographic conditions of Method A, of 16 to 20 minutes

Another aspect of the invention relates to a composition comprising a compound selected from the group consisting of a compound of Formula I, a compound of Formula II and a compound of Formula III, together with pharmaceutically acceptable excipients and carriers.

Preferably, the compounds which perform positively under the test conditions of Method 1, perform to a standard or level at least as well as a compound of Formula III. Thus, in another aspect, the present invention relates to the use of compound of Formula I or Formula II for binding to the urotensin II receptor wherein said compound, under the test conditions of Method 1, increases cellular growth to an extent greater than that of compound of Formula III. Accordingly, another aspect of the present invention relates to the use of compound of Formula I which, under the test conditions of Method 1, increases cellular growth to an extent greater than that of compound of Formula III or produces a detectable cellular response.

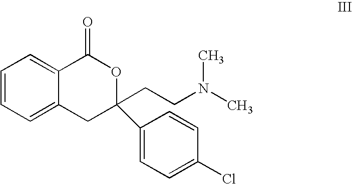

The functional potency of a compound may be measured in terms of its EC₅₀. Example I describes the performance of one embodiment of a compound of Formula I. The compound of Formula III has an EC₅₀ for the urotensin II receptor of 200 nM, with a maximal biological response in this system that is equal to or greater than the response evoked by the urotensin II peptide.

Remarkably, compounds of Formula I selectively modulate the urotensin II receptor, as shown in Example 1. The compounds had selectivity for the urotensin II receptor over several other receptors tested, including the CCKA receptor, acetylcholine receptors, serotonin 5 HT receptors, dopamine receptors, histamine receptors, and m3 muscarinic receptors.

The properties of the compounds of Formula I render it possible to alter cellular activity by specifically interacting with the human urotensin II receptor. Thus, another aspect of the invention relates to a method for augmenting cellular activity in a mammal, comprising activating the signalling of the urotensin II receptor, wherein activating the signalling is performed by the administration to the mammal of a substance binding to said receptor, the substance having an affinity for said receptor and the substance being administered in an amount effective to raise the concentration in the locality of the receptor of said substance to a level no lower than the affinity of said substance to the receptor, thereby effecting the binding of said substance to said receptor. The substance is, according to the present invention, a compound selected from the group comprising a compound of Formula I, a compound of Formula II and a compound of Formula III,

Moreover, the biological response induced by compounds of Formula I, as defined supra, allow for the use of said compounds as agonist in antagonist assays with urotensin II receptor. Furthermore, these biological responses produced as a result of the properties of compounds allows for the use of a compound of Formula I for the validation of the role of the urotensin II receptor as a drug target.

The finding that compounds of Formula I have potency for the human urotensin II receptor is quite unexpected. Many embodiments of the compounds of Formula I are novel and have not been associated to a form of treatment nor associated to a mode of action. Thus, one aspect of the present invention is these novel compounds of Formula II and their use as a medicament. This subclass of compounds of Formula I, termed Formula II, may act through the urotensin II receptor. It is also anticipated that this subclass of compounds will be useful as medicaments for ailments not necessarily associated with urotensin.

As stated, compounds of Formula I bind with high affinity and selectivity to the urotensin II receptor. One aspect of the invention relates to the use of compounds of Formula I for the preparation of a medicament for the treatment of diseases and disorders for which activation or modulation of the urotensin II receptor produces a physiologically beneficial response in a given disorder.

Compounds of the present invention may be used for the preparation of a medicament to modulate the activity of proteins or pathways that produce beneficial physiological effects in disease through modulation or alteration of signalling by the urotensin II receptor.

Accordingly, the invention further relates to a method of treating diseases or disorders in a mammal, said diseases and disorders requiring activation or modulation of the urotensin II receptor to produce a physiologically beneficial response in a given disease or disorder comprising administering an effective amount of a compound of Formula I. The diseases or disorders may be associated, for instance, with an imbalance of urotensin II and/or with an altered urotensin II receptor activity.

U.S. Pat. No. 3,880,885 and corresponding FR 72.11734 (Sandoz) disclose isocoumarins and isochromans as diuretics and hypotensives/antihypertensives. However, the art is completely silent as to the mechanism of action of the compounds. Antihypertension/hypotensives and diuretics may act through a variety of biological mechanisms. The understanding of the mode of action of compounds of Formula I contributes significantly to the art in that the medicaments, such as hypotensives/antihypertensives and diuretics, may now be prepared so as to target an identified cellular process or molecular target. The preparation may now be adapted so as to modulate the process or system within which the molecular target functions. This know-how may also be used for medicaments other than anti-hypertensive agents or diuretic agents, activating or modulating the urotensin II receptor to produce a physiologically beneficial response. Accordingly, the use of compound of Formula I for the preparation of a medicament for the treatment of diseases and disorders in a mammal requiring activation or modulation of the urotensin II receptor to produce a physiologically beneficial response in a given disease or disorder may be such that the medicament is not necessarily an anti-hypertensive/hypotensive agent or a diuretic agent.

As stated, one aspect of the present invention relates to the use of a compound selected from the group comprising a compound of Formula I, a compound of Formula II and a compound of Formula III, for the preparation of a medicament for the treatment of diseases and disorders for which activation or modulation of the urotensin II receptor produces a physiologically beneficial response in a given disorder. U.S. Pat. No. 3,880,885 discloses selected embodiments of Formula I for use as antihypertensive agents or diuretic agents. However, U.S. Pat. No. 3,880,885 is completely silent as to the mode of action of the selected compounds. The present invention provides understanding of the mode of action of compounds of Formula I, such as those disclosed in U.S. Pat. No. 3,880,885, thus allowing for a preparation of a medicament for the treatment of diseases and disorders for which activation or modulation of the urotensin II receptor produces a physiologically beneficial response. As stated, this understanding allows for a preparation to be manufactured and manufactured in such a way to target a biological pathway. Consequently, in a preferred embodiment the physiologically beneficial response is predominantly the result of the activation or modulation of the urotensin II receptor. Moreover, a further aspect of the present invention relates to a pharmaceutical composition comprising a compound of Formula I for the treatment of diseases and disorders for which activation or modulation of the urotensin II receptor produces a physiologically beneficial response in a given disorder. The composition may be adapted so as to specifically or selectively target and modulate the urotensin II receptor. Preferably, the physiologically beneficial response is predominantly the result of the activation or modulation of the urotensin II receptor.

Similarly, U.S. Pat. No. 4,564,641 discloses selected embodiments of Formula I for use in the treatment of mental disorders, in particular depression. However, U.S. Pat. No. 4,564,641 is completely silent as to the mode of action of the selected compounds. The present invention provides understanding of the mode of action of the compounds of Formula I, such as those disclosed in U.S. Pat. No. 4,564,641, thus allowing for a preparation of a medicament for the treatment of diseases and disorders for which activation or modulation of the urotensin II receptor produces a physiologically beneficial response. This understanding allows for a preparation to be manufactured in such a way to target a cellular process or molecular target.

A body of literature regarding the role of the pontine cholinergic nuclei and the modulation of cognitive processes has emerged in the last few years. Both basal forebrain and pontine cholinergic cell groups are known to control the activity of the hippocampal and cortical circuits that are critical for human attention, memory, and cognition (4). As such, the selective modulation of the activity of the PPT and LDTG nuclei present a novel pharmacological means to affect cognition and memory. Potential Disease States and Therapeutic Indications, Alzheimer's Disease and related dementias, schizophrenia and related psychoses.

In light of the distribution of the urotensin II receptor within the central nervous system and within cardiovascular tissue, it is anticipated that the compounds of Formula I will be useful as medicaments to treat an array of neurodegenerative, neuropsychiatric, neurological and cardiovascular disorders. Accordingly, a further aspect of the invention relates to the use of compound of Formula I for the preparation of a medicament for the treatment of diseases and disorders in a mammal selected from the group consisting of diseases and disorders associated with CNS function, such as Parkinson's Disease, Alzheimer's Disease, amylotrophic lateral sclerosis, muscular dystrophy, childhood spinal muscular atrophy, progressive spinal muscular atrophy and progressive bulbar palsy, OPCA, ADHD, schizophrenia, sleep disorders such as insomnia, and autonomic dysfunctions such as Shy Drager syndrome. In addition, compounds of Formula I may be useful as medicaments to treat cardiovascular disorders such as hypertension; hypotensive states related to shock, sepsis, major surgery and congestive heart failure.

As shown in Example 2, the present investigators have surprisingly found that compounds of Formula I had an effect on the motor function in mice whilst maintaining the traction reflex intact. That is to say that the mice were not anesthetized. Moreover, the alteration in locomotor activity occurred within a few minutes of administering the compound. The altered activity under the conditions of the experiment was a decrease in locomotor activity. Consequently, a further aspect of the invention relates to the use of compound of Formula I for the preparation of a medicament for altering the locomotor activity of a mammal, preferably such that the decrease in locomotor activity occurs within 10 minutes of administering the compound, such as within 7 minutes, preferably with 5 minutes, most preferably within 3, 2, or 1 minute. Correspondingly, the invention further relates to a method of altering the locomotor activity of a mammal, comprising administering to said mammal an effective amount of a compound of Formula I.

The decrease in locomotor activity and expression of urotensin II receptor in the brainstem are consistent with action of the compounds of Formula I on the CNS to alter sleep/wake patterns. The PPT and LDTG send ascending projections to the thalamus that are critical mediators of sleep and wakefulness in humans (2,3). During the sleep state, thalamocortical activity is dominated by rhythmic oscillations that are abolished during the transition to wakefulness, resulting in a significant increase in neuronal responsiveness. The cholinergic cells groups are one of the primary mediators of this transition, where neuronal activity of the PPT and LDTG neurons increase with wakefulness. Therefore, modulators of GPR-14 which can increase the activity of these cells may increase wakefulness in humans, while those that decrease the activity of these neurons may induce sleep. Consistent with these observations are the potential clinical use of modulators of GPR-14 as CNS stimulants and sleep promoting CNS depressants (both perhaps without the addictive and physical dependency properties that limit the use of current agents).

Thus, potential disease states and therapeutic indications for which compounds of Formula may be connected to include narcolepsy, non-addictive CNS Stimulant, ADHD and Insomnia Thus, another aspect of the invention, to the use of compound of Formula I for the preparation of a medicament for sleep disorders such as insomnia.

In light of the distribution of the receptor in cardiovascular tissue, the use of compound of Formula I for the preparation of a medicament acting through the activation of urotensin receptor II signalling for regulating blood pressure in a mammal is a particularly interesting aspect of the invention as well as the use of compound of Formula I for the preparation of a medicament acting through the activation of urotensin receptor II signalling for altering the heart rate or cardiac output in a mammal. Correspondingly, a method of altering the vascular pressure in a mammal, comprising constricting or dilating vascular tissue in said mammal, the constricting or dilating is performed by the activation of urotensin receptor signalling, said activation being performed by the administration of an effective amount compound of Formula I is anticipated. Moreover, method of altering the heart rate in a mammal, comprising the activation of a urotensin receptor, said activating being performed by the administration of an effective amount compound of Formula I is also anticipated.

Moreover, the use of compound of Formula I for the preparation of a diuretic agent acting through the activation of urotensin receptor II signalling is also anticipated.

A further aspect of the invention relates to a pharmaceutical composition comprising a compound of Formula I and pharmaceutically acceptable excipients or carriers formulated in a manner known to the skilled artisan such as according to formulations disclosed in Remington's Pharmaceutical Sciences. The composition may be formulated for oral administration, for administration via mucous membranes, or, amongst others parenteral administration in accordance with accepted practices.

### EXAMPLES

### Example 1

Receptor Selection and Amplification (R-SAT) Assays

The test compound of Formula III was screened according to a modification of the method described in U.S. Pat. No. 5,912,312. The method, herein referred to as Method 1, consists of the following:

Method 1

Receptor Selection and Amplification (R-SAT) Assays.

R-SAT assays were performed with minor modifications from that previously described (6). Briefly, NIH3T3 cells were grown in tissue culture treated rollerbottles to 40-50% confluence. Cells were transfected for 12-16 hours with plasmid DNAs using superfect (Qiagen Inc.) as per manufacture's protocols. R-SAT's were generally performed with 10 μg/rollerbottle of receptor and 50 μg/rollerbottle of Beta-galactosidase plasmid DNA. All receptor and G-protein constructs used were in the PSI mammalian expression vector (Promega Inc.). The transfected cells were then trypsinized and frozen in DMEM containing 10% DMSO. Frozen cells were later thawed, plated at 10,000-40,000 cells per well of a 96 ½ area plate that contained drug. Cells were then grown in a humidified atmosphere with 5% ambient CO₂ for five days. Media was then removed from the plates and marker gene activity was measured by the addition of the beta-galactosidase substrate ONPG (in PBS with 5% NP-40). The resulting colorimetric reaction was measured in a spectrophotometric plate reader (Titertek Inc.) at 420 nM.

TABLE 1

| | | | | | Competitive | |
|---|---|---|---|---|---|---|
| | Agonist | | Inverse Agonist | | Antagonist | |
| Target Name | EC50 | % Eff | IC50 | % Inh | Ki | % Inh |
| Urotensin II | 7 | 132 | | | | |
| Acetylcholine m1 | 2 | 3 | | | 2 | 13 |
| Acetylcholine m3 | 2 | 1.8 | | | 2 | 28 |
| Acetylcholine m5 | 2 | 3.6 | | | <5.65 | 55 |
| DopamineD2 | 2 | 13.6 | | | 2 | 13.2 |
| DopamineD1 | 2 | 0 | | | 2 | 0 |
| DopamineD5 | 2 | 0 | | | 2 | 0 |
| Serotonin 5HT-1E | 2 | 19 | | | | |
| Serotonin 5HT2a | 2 | 58 | 2 | 11.9 | | |
| Serotonin 5HT2b | 2 | 0 | 2 | 0 | 2 | 0 |
| Serotonin 5HT6 | 2 | 0 | | | | |
| Serotonin 5HT7 | 2 | 0 | | | | |
| Histamine H2 | 2 | 0 | 2 | 0 | | |
| Beta Adrenergic 1 | 2 | 0 | | | | |
| Beta Adrenergic 2 | 2 | 0 | | | 2 | 0 |
| SST2 | 2 | 0 | | | | |
| SST5 | 2 | 0 | | | | |
| SST3 | 2 | 0 | | | | |
| CRF1 | 2 | 0 | | | | |
| CRF2a | 2 | 0 | | | | |
| CRF2b | 2 | 0 | | | | |
| k opioid | 2 | 0 | | | | |
| CCKa | 2 | 0 | | | | |
| Serotonin 5HT-1A | | | | | 2 | 0 |
| Serotonin 5HT2c | | | 2 | 0 | | |
| Histamine H1 | | | | | 2 | 0 |

EC₅₀, IC₅₀ and Kᵢ are presented as the negative of the calculated potency in Molar. A value of “2” indicates that the potency in this case could not be calculated; % Eff is the percent maximal activation relative to control compound in each experiment; % Inh is the percent maximal inhibition relative to repression of basal activity by a control standard compound in each experimental case.

### Example 2

The test compound of Formula III was tested at 10 mg/kg, i.v. in male NSA mice. Locomotor activity was measured for 15 min following injection. The locomotor activity was decreased relative to sham injected animals. Within a few minutes into the session, the mice sat quietly in a corner of the chamber in a normal posture. Mice were responsive to sound and touch and otherwise appeared normal.

Following the initial experiment, a dose-response curves using 1, 3, and 10 mg/kg, i.v. were conducted. The effects of 10 mg/kg the test compound of Formula III were replicated.

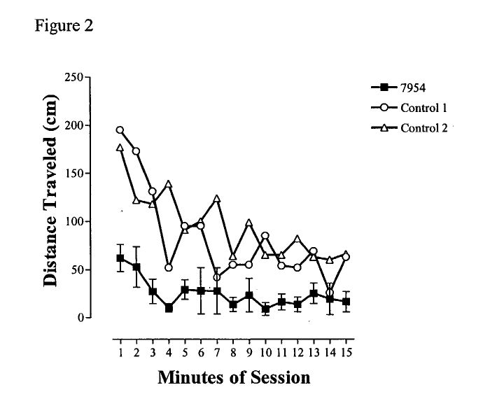

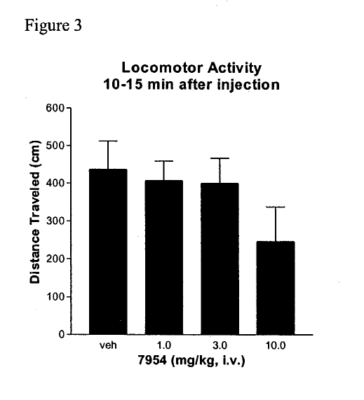

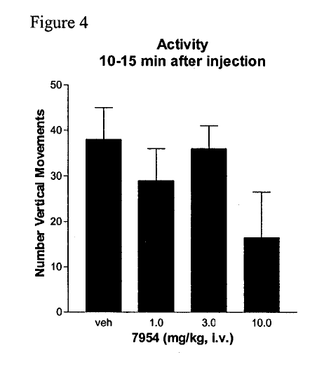

The behavioural experiments are shown in FIGS. 2-4. The results indicate CNS modulation by the test compound.

### Example 3

Synthesis of the Compounds of the Invention

Experimental Section

¹H and ¹³C spectra were recorded using Varian Unity 400 or Varian Unity 500 instruments. The solvent was in all cases CDCl₃. All reactions were followed by TLC (Merck silica gel 60 F₂₅₄) and analysed under UV (254 nm). In case of flash chromatography, Merck silica gel 60 (230-400 mesh) was used. Melting points were recorded on a Büchi melting point B-545 and are uncorrected. Gas chromatography analyses were performed on a Varian 3900 gas chromatograph equipped with a flame ionisation detector (FID). For the separation a fused silica column (CP5860) was used with hydrogen as carrier gas.

Amines

General Synthetic Procedure for the Mannich Reactions

Procedure A: The acetophenone (2 mmol), paraformaldehyde (2 mmol calculated on the monomer) and the amine hydrochloride (2 mmol) were dissolved in dioxane and heated to 200° C. in an Emrys Synthesizer™ for 300 s. The mixture was poured into saturated NaHCO₃ and extracted twice with EtOAc. An analytical sample for characterization was purified using flash chromatography (DCM/MeOH/TEA 94:5:1). The resulting product from procedure A was dissolved in ether and an HCl-saturated ether solution was added. After filtration the crystals were recrystallized from DCM/diethyl ether to afford the title compounds as white needles.

Procedure B: The acetophenone (40 mmol), paraformaldehyde (40 mmol, counted on the monomer) and the amine hydrochloride (40 mmol) were.dissolved in dioxane (100 mL) and heated in the large scale cavity. The mixture was poured into saturated NaHCO₃ and extracted twice with EtOAc, the crude product was purified using flash chromatography (DCM/MeOH/TEA 94:5:1). The resulting product was dissolved in ether and an HCl-saturated ether solution was added. After filtration the crystals were recrystallized from DCM/diethyl ether to afford the title compounds as white needles.

1-(2-Chlorophenyl)-3-dimethylamino-propan-1-one hydrochloride

(B) Yielded 41% when run at a 40 mmol scale; ¹H NMR (400 MHz) δ2.77 (s, 3H), 2.81 (s, 3H), 3.42-3.47 (m, 2H), 3.67 (t, 2H, J=6.8 Hz), 7.28-7.32 (m, 1H), 7.36-7.38 (m, 1H), 7.57 (d, 2H, J=7.6 Hz), 12.35 (s, 1H). ¹³C NMR (100 MHz) δ37.6, 43.3 (2 C:s), 52.4, 127.2, 129.6, 130.9, 131.3, 132.9, 136.8, 197.9. Mp 163.5-165.2° C.

1-(3-Chlorophenyl)-3-dimethylaminopropan-1-one hydrochloride

(B) Yielded 48% when run at a 40 mmol scale ¹H NMR (400 MHz) [5] δ3.09 (s, 6H), 3.47-3.52 (m, 2H), 3.70 (t, 2H, J=6.8 Hz), 6.89 (t, 2H, J=6.8 Hz), 7.71-7.74 (m, 1H), 7.80-7.88 (m, 2H), 8.13-8.21 (m, 1H), 12.35 (s, 1H). ¹³C NMR (100 MHz) δ34.0, 43.4 (2 C:s), 52.6, 126.5, 128.2, 130.3, 134.1, 135.3, 136.7, 194.5. Mp 191.8-192.6° C.

1-(4-Chlorophenyl)-3-dimethylamino-propan-1-one hydrochloride

(A) Yield: 49% (LC/MS); ¹H NMR (400 MHz) δ2.85 (s, 6H), 3.50 (t, 2H, J=7.2 Hz), 3.74 (t, 2H, J=7.2 Hz), 7.45 (dd, 2H, J=8.4, 2.0 Hz), 7.99 (dd, 2H, J=8.4, 2.0 Hz), 12.30 (s, 1H). ¹³C NMR (100 MHz) δ33.8, 43.3 (2 C:s), 52.7, 129.2, 129.6 (2 C:s), 133.6 (2 C:s), 140.7, 194.5. Mp 172.5-173.5° C.

3-Diethylamino-1-(3-fluorophenyl)-propan-1-one

(B) Yielded 35% when run at a 40 mmol scale as a pale yellow oil. ¹H NMR (400 MHz) δ2.86 (s, 6H), 3.45-3.53 (m, 2H), 3.76-3.79 (m, 2H), 7.32-7.34 (m, 1H), 7.47-7.52 (m, 1H), 6.67 (d, 1H, J=8.0 Hz), 7.81 (d, 1H, J=7.6 Hz). ¹³C (100 MHz) δ34.2, 43.6 (2 C:s), 52.8, 115.1 (d, ¹J_{CF}=120 Hz), 120.3, 120.9, 121.5 (d, ²J_{CF}=65.5 Hz), 124.4, 130.8 (d, ²J_{CF}=35 Hz), 194.6.

3-Diethylamino-1-(4-fluorophenyl)-propan-1-one

(B) Yielded 28% when run at a 40 mmol scale as a pale yellow oil. ¹H NMR (400 MHz) δ2.86 (s, 6H), 3.51 (t, 2H, J=7.0 Hz), 3.75 (t, 2H, J=7.0 Hz), 7.14-7.17 (m, 2H), 8.00-8.05 (m, 2H). ¹³C (100 MHz) δ33.9, 43.6 (2 C:s), 52.9, 116.2 (d, 2 C:s, ²J_{CF}=88 Hz), 131.3 (d, 2 C:s, ³J_{CF}=38 Hz), 132.1, 166.5 (d, ¹J_{CF}=202 Hz), 194.4.

3-Dimethylamino-1-(2-methoxyphenyl)-propan-1-one

(A) Yield 30% (LC/MS)(pale yellow oil); ¹H NMR (400 MHz) [6] δ2.79 (s, 6H), 3.43 (t, 2H, J=8.5 Hz), 3.69 (t, 2H, J=8.5 Hz), 3.98 (s, 3H), 6.99-7.04 (m, 2H), 7.50-7.55 (m, 1H), 7.72-7.75 (m, 1H). ¹³C NMR (125 MHz) δ39.1, 43.4 (2 C:s), 52.7, 52.8, 111.7, 120.7, 121.2, 130.5, 134.7, 159.1, 197.6.

3-Dimethylamino-1-(3-methoxyphenyl)-propan-1-one hydrochloride

(A) Yield 61% (LC/MS); ¹H NMR (400 MHz) [7] δ2.85 (s, 6H), 3.51 (t, 2H, J=6.8 Hz), 3.73 (t, 2H, J=6.8 Hz), 3.84 (s, 3H), 7.12-7.16 (m, 1H), 7.37 (t, 1H, J=8.4 Hz), 7.45-7.46 (m, 1H), 7.56-7.58 (m, 1H), 12.50 (s, 1H). ¹³C NMR (100 MHz) δ33.8, 43.3 (2 C:s), 52.7, 55.5, 112.2, 120.6, 120.9, 129.9, 136.6, 159.9, 195.6. Mp 153.0-154.0° C.

3-Dimethylamino-1-(4-methoxyphenyl)-propan-1-one hydrochloride

(B) Yielded 31% when run at a 40 mmol scale ¹H NMR (400 MHz) δ2.81 (s, 3H), 2.83 (s, 3H), 3.46-3.52 (m, 2H), 3.66 (t, 2H, J=6.8 Hz), 3.85 (s, 3H), 6.91 (d, 2H, J=8.0 Hz), 7.94 (d, 2H, J=8.0 Hz), 12.60 (s, 1H). ¹³C NMR (100 MHz) δ33.2, 43.2 (2 C:s), 52.6, 55.4, 114.2 (2 C:s), 128.5, 130.8 (2 C:s), 164.2, 194.3. Mp 150.2-152.1° C.

3-Dimethylamino-1-(4-methylphenyl)-propan-1-one hydrochloride

(B) Yield 59% when run at a 40 mmol scale; 1H NMR (400 MHz) δ2.35 (s, 3H), 2.76 (s, 3H), 2.80 (s, 3H), 3.44-3.47 (m, 2H), 3.65 (t, 2H, J=7.2 Hz), 7.21 (d, 2H, J=8.1 Hz), 7.82 (d, 2H, J=8.0 Hz), 12.40 (s, 1H). ¹³C NMR (100 MHz) δ21.7, 33.6, 43.3 (2 C:s), 52.7, 129.4, 129.5 (2 C:s), 132.8 (2 C:s), 145.2, 195.2. Mp 160.4-160.7° C.

3-Diethylamino-1-(1-naphtyl)-propan-1-one hydrochloride

(B) Yielded 38% when run at a 40 mmol scale. ¹H NMR (400 MHz) δ2.87 (d, 6H, J=5.1 Hz), 3.54-3.59 (m, 2H), 3.87 (d, 2H, J=7.0 Hz), 7.51-7.63 (m, 3H), 7.89 (d, 1H, J=7.3 Hz), 8.05 (d, 1H, J=8.4 Hz), 8.12 (d, 1H, J=7.4 Hz), 8.71 (d, 1H, J=8.8 Hz), 12.90 (s, 1H). ¹³C (100 MHz) δ36.3, 43.4 (2 C:s), 52.9, 124.5, 125.3, 126.6, 128.5, 128.7, 129.7, 130.1, 132.9, 133.9, 134.4, 195.2.

3-Diethylamino-1-(2-naphtyl)-propan-1-one hydrochloride

(B) Yielded 53% when run at a 40 mmol scale. ¹H NMR (400 MHz) δ2.87 (s, 6H), 3.57 (t, 2H, J=7.2 Hz), 3.89 (t, 2H, J=7.2 Hz), 7.56-7.65 (m, 2H), 7.86-7.91 (m, 2H), 7.99-8.02 (m, 2H), 8.56 (s, 1H), 13.00 (s, 1H). ¹³C (100 MHz) δ33.8, 43.3 (2 C:s), 52.8, 123.2, 127.1, 127.7, 128.8, 129.1, 129.8, 130.6, 132.3, 132.6, 135.9, 195.5.

3-Dimethylamino-1-(4-phenoxyphenyl)-propan-1-one hydrochloride

(B) Yielded 59% when run at a 40 mmol scale. ¹H NMR (400 MHz) δ2.80 (s, 3H), 2.88 (s, 3H), 3.07-3.11 (m, 2H), 3.69 (t, 2H, J=6.8 Hz), 6.98 (d, 2H, J=8.8 Hz), 7.05 (d, 2H, J=7.6 Hz), 7.19-7.21 (m, 1H), 7.37-7.41 (m, 2H), 7.96 (d, 2H, J=8.8 Hz), 12.40 (s, 1H). ¹³C NMR (100 MHz) δ33.5, 43.3 (2 C:s), 52.7, 117.3, 120.3, 124.9, 129.9 (2 C:s), 130.1 (2 C:s), 130.6, 155.0, 162.8, 194.1. Mp 148.1-149.6° C.

3-Dimethylamino-1-phenyl-propan-1-one hydrochloride

(B) Yield: 40% when run at a 40 mmol scale; ¹H NMR (400 MHz) δ2.81 (s, 3H), 2.89 (s, 3H), 3.50-3.54 (m, 2H), 3.75 (t, 2H, J=6.8 Hz), 7.47 (t, 2H, J=7.6 Hz), 7.58-7.62 (m, 1H), 7.98 (dd, 2H, J=1.2, 8.4 Hz), 12.8 (s, 1H). ¹³C NMR (100 MHz) δ33.8, 43.4 (2 C:s), 52.7, 128.2, 128.8 (2 C:s), 134.1(2 C:s), 135.3, 195.7. Mp 153.6-153.7° C.

3-Diethylamino-1-(2-thiophenyl)-propan-1-one hydrochloride

(B) Yielded 48% when run at a 40 mmol scale. ¹H NMR (400 MHz) δ2.82 (s, 6H), 3.48 (t, 2H, J=7.0 Hz), 3.69 (t, 2H, J=7.0 Hz), 7.17 (dd, 1H, J=8.6, 9.2 Hz), 7.72 (d, 1H, J=9.2 Hz), 7.89 (d, 1H, J=8.6 Hz). ¹³C (100 MHz) δ34.2, 43.2 (2 C:s), 52.3, 128.8, 133.7, 135.2, 142.4, 188.6.

3-Dimethylamino-1-(4-trifluoromethyl-phenyl)-propan-1-one hydrochloride

(B) Yield 69% when run at a 40 mmol scale ¹H NMR (400 MHz) δ2.89 (s, 6H), 3.54 (t, 2H, J=6.8 Hz), 3.83 (t, 2H, J=6.8 Hz), 7.51 (d, 2H, J=7.6 Hz), 8.13 (d, 2H, J=7.6 Hz), 12.40 (s, 1H). ¹³C NMR (100 MHz) δ34.2, 43.5, 52.7, 123.4 (q, ¹J_{CF}=135 Hz), 125.9 (q, ²J_{CF}=15 Hz), 128.7, 135.3, 137.9, 194.8. Mp 151.0-152.3° C.

General Procedure II

3,4′-Dichloropropiophenone was dissolved in THF and the secondary amine (2 eq.) was added. After stirring for 12 h, the mixture was poured into saturated aqueous NH₄Cl and extracted twice with EtOAc. The combined organic phases were washed (H₂O and brine) and evaporated. The crude oil was dissolved in diethyl ether and HClₑₜₕₑᵣ was added. The resulting solid was recrystallized from CH₂Cl₂/diethyl ether to afford the title compounds.

1-(4-Chlorophenyl)-3-(piperidin-1-yl)-propan-1-one HCl

3,4′-Dichloropropiophenone (6.0 g, 30 mmol) and piperidine (4.9 g, 60 mmol) yielded 6.7 g (80% yield) of the title compound as white crystals. Mp 194.2-194.8° C. ¹H NMR (400 MHz) δ1.38-1.45 (m, 1H), 1.78-1.82 (m, 3H), 2.16-2.26 (m, 2H), 2.65-2.74 (m, 2H), 3.37-3.40 (m, 2H), 3.48-3.51 (m, 2H), 3.78 (t, 2H, J=6.8 Hz), 7.40 (d, 2H, J=8.4 Hz), 7.90 (d, 2H, J=8.4 Hz), 12.10 (s, 1H). ¹³C NMR (100 MHz) δ21.9, 22.6 (2 C:s), 33.3, 51.9, 53.8 (2 C:s), 129.1, 129.7 (2 C:s), 133.8 (2 C:s), 140.5, 195.0.

1-(4-Chlorophenyl)-3-(pyrrolidin-1-yl)-propan-1-one HCl

3,4′-Dichloropropiophenone (8.0 g, 39.4 mmol) and pyrrolidine (5.6 g, 78.8 mmol) yielded 3.0 g (32% yield) of the title compound as white crystals. Mp 184.2-184.8° C. ¹H NMR (500 MHz) δ2.09-2.13 (m, 2H), 2.22-2.26 (m, 2H), 2.83-2.88 (m, 2H), 3.51-3.55 (m, 2H), 3.71-3.78 (m, 4H), 7.45 (d, 2H, J=8.5 Hz), 7.93 (d, 2H, J=8.5 Hz), 12.75 (s, 1H). ¹³C NMR (125 MHz) δ23.4, 23.7, 34.9, 50.1, 54.1, 54.2, 129.5, 130.1 (2 C:s), 134.0 (2 C:s), 141.0, 194.1.

1-(4-Chlorophenyl)-3-(morpholin-1-yl)-propan-1-one HCl

3,4′-Dichloropropiophenone (6.0 g, 30 mmol) and morpholine (5.2 g, 60 mmol) yielded 7.5 g (99% yield) of the title compound as white crystals. Mp 85.8-86.2° C. ¹H NMR (400 MHz) δ2.49 (t, 4H, J=4.4 Hz), 2.80 (t, 2H, J=7.6 Hz), 3.14 (t, 2H, J=7.6 Hz), 3.69 (t, 4H, J=4.4 Hz), 7.42 (d, 2H, J=6.8 Hz), 7.88 (d, 2H, J=6.8 Hz), 12.10 (s, 1H). ¹³C NMR (100 MHz) δ35.9 (2 C:s), 53.4, 53.6, 66.8 (2 C:s), 128.9, 129.4 (2 C:s), 135.0 (2 C:s), 139.5, 197.6.

1-(4-Chlorophenyl)-3-(4-methyl-piperazin-1-yl)-propan-1-one

3,4′-Dichloropropiophenone (6.0 g, 30 mmol) and 4-methyl-piperazine (5.2 g, 51.9 mmol) yielded 8.5 g (98% yield) of the title compound as white crystals. Mp 69.2-69.7° C. ¹H NMR (400 MHz) δ2.27 (s, 3H), 2.31-2.61 (m, 8H), 2.82 (t, 2H, J=7.1 Hz), 3.12 (t, 2H, J=7.1 Hz), 7.41 (d, 2H, J=8.0 Hz), 7.88 (d, 2H, J=8.0 Hz). ¹³C NMR (100 MHz) δ36.2, 45.9 (2 C:s), 52.9, 53.2 (2 C:s), 55.0, 128.8 (2 C:s), 129.4 (2 C:s), 135.1, 139.5, 197.8.

1-(4-Chlorophenyl)-3-diethylamino-propan-1-one

3,4′Dichloropropiophenone (3.0 g, 15 mmol) and diethylamine (2.2 g, 30 mmol) yielded 3.1 g (75% yield) of the title compound as white crystals. ¹H NMR (500 MHz) δ1.41 (t, 6H, J=7.5 Hz), 3.01-3.13 (m, 2H), 3.18-3.21 (m, 2H), 3.41-3.45 (m, 2H), 3.76-3.79 (m, 2H), 7.43 (d, 2H, J=8.5 Hz), 7.93 (d, 2H, J=8.5 Hz), 12.30 (s, 1H). ¹³C NMR (125 MHz) δ8.3, 33.5, 46.7, 47.1, 129.1 (2 C:s), 129.7 (2 C:s), 133.8, 140.6, 194.9.

2,N-Dimethyl-benzamides

General Procedure for the Preparation of 2,N-dimethyl-benzamides

The benzoic acid was dissolved in THF (75 mL/g) and triethylamine (5 eqv) was added. Under vigorous stirring SOCl₂ (1.3 eq.) was added drop wise and the mixture was stirred at rt for 20 minutes. Methylamine (2M in THF) (2 eqv) was added slowly and the reaction was stirred for another 2 h. The mixture was poured into water and extracted twice with EtOAc. After concentration of the combined organic phases the crude oil was dissolved in CH₂Cl₂ and filtered through a plug of silica/MgSO₄ (5:1) Evaporation of the solvent yielded the pure amides.

2,3,N-Trimethyl benzamide

2,3-Dimethylbenzoic acid (3 g; 20 mmol) yielded 2g (63%) of the title compound as light yellow crystals. Mp 99.0-99.1° C. ¹H NMR (400 MHz) δ2.20 (s, 3H), 2.22 (s, 3H), 2.90 (d, 3H, J=4.8 Hz), 5.70 (bs, 1H), 7.00-7.11 (m, 2H), 7.18 (d, 1H, J=3.6 Hz). ¹³C NMR (100 MHz) δ16.1, 20.1, 26.4, 124.1, 125.2, 130.8, 133.8, 137.2, 137.5, 171.5. Anal. Calcd for C₁₀H₁₃NO: C, 73.6; H, 8.0; N, 8.6. Found: C, 73.4; H, 8.0; N, 8.4.

2,4,N-Trimethyl benzamide

2,4-Dimethylbenzoic acid (3 g; 20 mmol) yielded 3.0 g (92%) of the title compound as light yellow crystals. Mp 95.9-96.1° C. ¹H NMR (400 MHz) δ2.42 (s, 3H), 2.48 (s, 3H), 2.97 (d, 3H, J=7.5 Hz), 5.80 (bs, 1H), 6.98 (d, 1H, J=7.7 Hz), 7.01 (s, 1H), 7.24 (d, 1H, J=7.7 Hz). ¹³C NMR (100 MHz) δ19.8, 21.2, 26.6, 126.2, 126.7, 131.7, 133.5, 136.1, 139.8, 170.8

2,5,N-Trimethyl benzamide

2,5-Dimethylbenzoic acid (3 g; 20 mmol) yielded 2.9 g (89%) of the title compound as light yellow crystals. Mp 119.0-119.7° C. ¹H NMR (400 MHz) δ2.29 (s, 3H), 2.37 (s, 3H), 2.96 (d, 3H, J=7.6 Hz), 5.80 (bs, 1H), 7.08-7.11 (m, 2H), 7.14 (s, 1H). ¹³C NMR (100 MHz) δ19.2, 20.7, 26.5, 127.2, 130.3, 130.7, 132.6, 135.1, 136.2, 171.0

3-Methoxy-2,N dimethyl-benzamide. (F118) (FL:21)

3-Methoxy-2-methyl-benzoic acid (3.5 g; 21.0 mmol) yielded 3.7 g (99%) of the title compound as light yellow crystals. Mp 107.1-107.6° C. ¹H NMR (500 MHz) δ2.32 (s, 3H), 2.99 (d, 3H, J=5.0 Hz), 3.85 (s, 3H), 5.89 (bs, 1H), 6.80-6.95 (m, 2H), 7.10-7.16 (m, 1H). ¹³C NMR (125 MHz) δ12.5, 26.5, 55.6, 111.2, 118.6, 124.5, 126.5, 138.2, 157.9, 170.7

3-Fluoro-2, N dimethyl-benzamide. (F125) (FL:23)

3-Fluoro-2-methyl-benzoic acid (3.0 g; 19.4 mmol) yielded 3.1 g (96%) of the title compound as light yellow crystals. Mp 91.5-92.4° C. ¹H NMR (500 MHz) δ2.26 (s, 3H), 2.92 (d, 3H, J=4.5 Hz), 6.08 (bs, 1H), 7.00-7.13 (m, 3H). ¹³C NMR (125 MHz) δ11.2 (d, ³J_{CF}=4.6 Hz), 26.5, 116.4 (d, ²J_{CF}=23 Hz), 122.1, 123.3 (d, ²J_{CF}=18 Hz), 126.9 (d, ³J_{CF}=8.9 Hz), 138.8 (d, ³J_{CF}4.1 Hz), 161.3 (d, ¹J_{CF}=244 Hz), 169.5

Isocoumarines

General Procedure for the Synthesis of Isocoumarines

The benzamide was dissolved in THF (15 mL/g) and n-BuLi (2.2 eq.) was added slowly at rt. After 1 h, the ketone (0.5 eq.) was added to the intense red solution and the mixture was stirred over night. The reaction was poured into a saturated aqueous NH₄Cl solution (twice the THF volume) and extracted twice with EtOAc. The combined organic phases were washed with brine and evaporated. The crude oil was dissolved in 1,2-dichlorobenzene and heated to 105° C. for 48 h. After cooling, the mixture was diluted with CH₂Cl₂ and applied directly to a flash column. After flash chromatography (94% CH₂Cl₂, 5% MeOH, 1% TEA, rf=0.3) the fractions containing product were evaporated and the resulting oil dissolved in diethyl ether. After filtration of the solution HClₑₜₕₑᵣ was added and the resulting crystals recrystalized from CH₂Cl₂/diethyl ether to afford the title compounds as white solids.

3-(2-Dimethylaminoethyl)-3-phenyl-isochroman-1-one HCl (F70) (FL8)

2,N-Dimethyl-benzamide (1 g, 6.7 mmol) and 3-dimethylamino-propiophenone yielded 100 mg (10%) of the title compound. Mp>250° C. (decomp.). ¹H NMR (400 MHz) δ2.63-2.81 (m, 9H), 3.18-3.26 (m, 1H), 3.50 (s, 2H), 7.14, (d, 1H, J=7.6 Hz), 7.19-7.22 (m, 1H), 7.26-7.35 (m, 5H), 7.44 (dt, 1H, J=1.2, 7.6 Hz), 7.97 (d, 1H, J=7.6 Hz) 12.30 (bs, 1H). ¹³C NMR (100 MHz) δ36.5 (2 C:s), 39.1, 42.3, 53.3, 83.9, 124.4, 124.7 (2 C:s), 127.8, 127.9, 128.2, 129.1 (2 C:s), 129.9, 134.4, 136.5, 139.6, 164.3. Anal. Calcd for C₁₉H₂₂ClNO₂: C, 68.8; H, 6.7; N, 4.2. Found: C, 68.5; H, 6.7; N, 4.2.

3-(2-Dimethylaminoethyl)-5-methyl-3-phenyl-isochroman-1-one HCl (F82) (FL13)

2,3,N-Trimethyl-benzamide (1 g, 6.1 mmol) and 3-dimethylamino-propiophenone yielded 250 mg (24%) of the title compound. Mp>250° C. (decomp.). ¹H NMR (400 MHz) δ2.27 (s, 3H), 2.65-2.83 (m, 9H), 3.18-3.22 (m, 1H), 3.31 (d, 1H, ²J=16.4 Hz), 3.52 (d, 1H, J=16.4 Hz), 7.16-7.36 (m, 7H), 7.86 (d, 1H, J=7.6 Hz), 12.30 (bs, 1H). ¹³C NMR (100 MHz) δ18.9, 36.2, 36.6, 42.5, 44.0, 53.4, 83.3, 124.4 (2 C:s), 124.6, 127.3, 127.8, 128.3, 129.2 (2 C:s), 135.1, 135.5, 135.8, 140.0, 164.8. HRTofMS calcd for C₂₀H₂₄NO₂ (M+H) m/z 310.1807, found 310.1811

3-(2-Dimethylaminoethyl)-7-methyl-3-phenyl-isochroman-1-one HCl (F87) (FL15)

2,5,N-Trimethyl-benzamide (1 g, 6.1 mmol) and 3-dimethylamino-propiophenone yielded 250 mg (24%) of the title compound. Mp>250° C. (decomp.). ¹H NMR (400 MHz) δ2.30 (s, 3H), 2.64-2.81 (m, 9H), 3.16-3.22 (m, 1H), 3.46 (s, 2H), 7.04 (d, 1H, J=7.6 Hz), 7.20-7.36 (m, 6H), 7.80 (s, 1H), 12.25 (bs, 1H). ¹³C NMR (100 MHz) δ20.9, 36.5, 38.8, 42.6, 43.9, 53.4, 84.0, 124.1, 124.7 (2 C:s), 127.7, 128.3, 129.1 (2 C:s), 130.2, 133.5, 135.4, 137.8, 139.8, 164.7. HRTofMS calcd for C₂₀H₂₄NO₂ (M+H) m/z 310.1807, found 310.1826

3-(2-Dimethylaminoethyl)-6-methyl-3-phenyl-isochroman-1-one HCl (F102) (F24)

2,4,N-Trimethyl-benzamide (1 g, 6.1 mmol) and 3-dimethylamino-propiophenone yielded 200 mg (19%) of the title compound. Mp>250° C. (decomp.). ¹H NMR (500 MHz) δ2.31 (s, 3H), 2.63-2.80 (m, 9H), 3.15-3.22 (m, 1H), 3.40-3.48 (m, 2H), 6.94 (s, 1H), 7.08 (d, 1H, J=8.0 Hz), 7.21-7.35 (m, 5H), 7.86 (d, 1H, J=8.0 Hz), 12.40 (bs, 1H). ¹³C NMR (125 MHz) δ21.7, 36.4, 39.2, 42.5, 43.8, 53.4, 83.8, 121.7, 124.8 (2 C:s), 128.2, 128.4, 128.8 (2 C:s), 129.1, 130.0, 136.6, 139.9, 145.6, 164.5. Anal. Calcd for C₂₀H₂₄ClNO₂: C, 69.5; H, 7.0; N, 4.0. Found: C, 69.4; H, 7.1; N, 4.0.

3-(2-Dimethylaminoethyl)-5-methoxy-3-phenyl-isochroman-1-one HCl (F120) (FL26)

3-Methoxy-2,N-dimethyl-benzamide (1 g, 5.6 mmol) and 3-dimethylamino-propiophenone yielded 175 mg (19%) of the title compound. Mp>250° C. (decomp.). ¹H NMR (500 MHz) δ2.62-2.80 (m, 9H), 3.14-3.19 (m, 1H), 3.28 (d, 1H, ²J=17 Hz), 3.64 (d, 1H, ²J=17 Hz), 3.83 (s, 3H), 7.01 (d, 1H, J=8.0 Hz), 7.23-7.39 (m, 6H), 7.61 (d, 1H, 8.0 Hz), 12.35 (bs, 1H). ¹³C NMR (125 MHz) δ32.6, 36.4, 42.5, 43.8, 53.4, 55.8, 83.7, 115.5, 121.4, 124.7 (2 C:s), 125.3, 125.5, 128.1, 128.2, 129.1 (2 C:s), 140.2, 155.9, 164.4. HRTofMS calcd for C₂₀H₂₄NO₃ (M+H) m/z 326.1756, found 326.1760.

3-(2-Dimethylaminoethyl)-5-fluoro-3-phenyl-isochroman-1-one HCl (F133) (FL33)

3-Fluoro-2,N-dimethyl-benzamide (1 g, 6.0 mmol) and 3-dimethylamino-propiophenone yielded 125 mg (6%) of the title compound. Mp>250° C. (decomp.). ¹H NMR (500 MHz) δ2.66-2.81 (m, 9H), 3.20-3.25 (m, 1H), 3.37 (d, 1H, ²J=16.5 Hz), 3.70 (d, 1H, ²J=16.5 Hz), 7.20-7.33 (m, 7H), 7.80 (d, 1H, J=8.0 Hz), 12.60 (bs, 1H). ¹³C NMR (125 MHz) δ31.9, 36.7, 42.7, 43.7, 53.2, 83.9, 120.9, 121.1, 123.8 (d, ²J_{CF}=25.5 Hz), 124.7 (2 C:s), 125.7, 126.2, 128.6 (d, ²J_{CF}=25.5 Hz) 129.3 (2 C:s), 139.4, 158.9 (d, ¹J_{CF}=246.6 Hz), 163.3. Anal. Calcd for C₁₉H₂₁ClFNO₂: C, 65.2; H, 6.1; N, 4.0. Found: C, 65.0; H, 6.1; N, 4.2.

3-(4-Chlorophenyl)-3-[2-(pyrrolidin-1-yl)-ethyl]-isochroman-1-one HCl (F162) (FL39)

2,N-Dimethyl benzamide (1 g, 6.7 mmol) and 4′-chloro-2-pyrrolidine-propiophenone yielded 340 mg (24%) of the title compound. Mp>250° C. (decomp.). 1H NMR (400 MHz) δ1.66-1.77 (m, 4H), 2.10-2.18 (m, 3H), 2.30-2.38 (m, 4H), 2.44-2.47 (m, 1H), 3.40-3.46 (m, 2H), 7.11-7.30 (m, 6H), 7.40-7.45 (m, 1H), 7.96 (d, 1H, J=8.0 Hz), 12.10 (bs, 1H). ¹³C NMR (100 MHz) δ23.3 (2 C:s), 38.0, 41.2, 50.5, 54.1 (2 C:s 84.8, 125.0, 126.6 (2 C:s), 127.6, 127.7, 128.6 (2 C:s), 129.9, 133.3, 134.0, 137.3, 140.4, 164.7. HRTofMS calcd for C₂₁H₂₃ClNO₂ (M+H) m/z 356.1417, found 356.1421

3-(4-Chlorophenyl)-3-(2-dimethylaminoethyl)-6-methyl-isochroman-1-one HCl (F170) (FL40)

2,4,N-Trimethyl benzamide (1 g, 6.1 mmol) and 4′-chloro-2-dimethylamino-propiophenone yielded 180 mg (20%) of the title compound. Mp>250° C. (decomp.). ¹H NMR (400 MHz) δ2.31 (s, 3H), 2.65-2.82 (m, 9H), 3.15-3.22 (m, 1H), 3.37-3.48 (m, 2H), 7.04 (d, 1H, J=8.0 Hz), 7.26-7.31 (m, 6H), 7.80 (s, 1 H), 12.30 (bs, 1H). ¹³C NMR (100 MHz) δ21.0, 36.3, 38.8, 42.5, 44.0, 53.3, 83.6, 123.9, 126.3 (2 C:s), 127.7, 129.3 (2 C:s), 130.3, 133.3, 134.3, 135.4, 138.0, 138.4, 164.4. Anal. Calcd for C₂₀H₂₃Cl₂NO₂* 0.25 H₂O: C, 62.4; H, 6.2; N, 3.6. Found: C, 62.6; H, 6.0; N, 3.7.

3-(4-Chlorophenyl)-3-[2-(piperidin-1-yl)-ethyl]-isochroman-1-one HCl (F173) (FL43)

2,N-Dimethyl benzamide (1 g, 6.7 mmol) and 4′-chloro-2-piperidine-propiophenone yielded 120 mg (8%) of the title compound. Mp>250° C. (decomp.). ¹H NMR (400 MHz) δ1.29-1.32 (m, 1H), 1.67-1.82 (m, 3H), 2.13-2.20 (m, 2H), 2.40-2.53 (m, 2H), 2.63-2.69 (m, 2H), 2.88-2.93 (m, 1H), 3.01-3.08 (m, 1H), 3.23-3.26 (m, 1H), 3.35-3.50 (m, 3H), 7.09 (d, 1H, J=7.2 Hz), 7.20-7.29 (m, 5H), 7.41-7.45 (m, 1H), 7.95 (dd, 1H, J=8.0, 0.8 Hz), 12.10 (bs, 1H). ¹³C NMR (100 MHz) δ22.0, 22.4, 22.5, 35.2, 39.2, 52.7, 52.9, 54.6, 83.9, 124.2, 126.4 (2 C:s), 127.9, 128.0, 129.3 (2 C:s), 130.0, 134.2, 134.6, 136.4, 138.5, 164.2. HRTofMS calcd for C₂₂H₂₅ClNO₂ (M+H) m/z 370.1574, found 370.1576.

3-(4-Chlorophenyl)-3-(2-dimethylaminoethyl)-5-methoxy-isochroman-1-one HCl (F174) (FL44)

3-Methoxy-2, N-dimethyl-benzamide (1 g, 5.6 mmol) and 4′-chloro-2-dimethylamino-propiophenone yielded 130 mg (13%) of the title compound. Mp>250° C. (decomp.). ¹H NMR (400 MHz) δ2.58-2.76 (m, 9H), 3.01-3.12 (m, 1H), 3.21 (d, 1H, ²J=17.2 Hz), 3.53 (d, 1H, ²J=17.2 Hz), 3.77 (s, 3H), 6.96 (dd, 1H, J=8.4, 1.2 Hz), 7.20-7.28 (m, 5H), 7.54 (dd, 1H, J=8.0, 0.8 Hz), 12.50 (bs, 1H). ¹³C NMR (100 MHz) δ32.5, 36.2, 42.4, 43.9, 53.3, 55.8, 83.4, 115.6, 121.4, 125.0, 125.1, 126.2 (2 C:s), 128.3, 129.3 (2 C:s), 134.2, 138.8, 155.8, 164.1. Anal. Calcd for C₂₀H₂₃Cl₂NO₃* 0.5 H₂O: C, 59.3; H, 6.0; N, 3.5. Found: C, 59.6; H, 5.8; N, 3.6.

3-(4-Chlorophenyl)-3-[2-(morpholin-1-yl)-ethyl]-isochroman-1-one HCl (F171) (FL45)

2,N-Dimethyl-benzamide (1 g, 6.7 mmol) and 4′-chloro-2-morpholin-propiophenone yielded 30 mg (3%) of the title compound. Mp>250° C. (decomp.). ¹H NMR (500 MHz) δ2.69-2.84 (m, 4H), 2.93-2.97 (m, 1H), 3.12-3.21 (m, 2H), 3.44-3.49 (m, 3H), 3.89-3.99 (m, 2H), 4.12-4.26 (m, 2H), 7.17 (d, 1H, J=7.5 Hz), 7.30-7.36 (m, 5H), 7.51 (dd, 1H, J=8.0, 7.5 Hz), 8.02 (d, 1H, J=8.0 Hz), 13.20 (bs, 1H). ¹³C NMR (125 MHz) δ34.9, 39.3, 51.6, 53.0, 53.2, 63.5 (2 C:s), 83.7, 124.2, 126.4 (2 C:s), 127.9, 128.1, 129.4 (2 H, 5.9; N, 3.3. Found: C, 59.1; H, 5.7; N, 3.5.

3-(4-Chlorophenyl)-3-[2-(4-methyl-piperazin-1-yl)-ethyl]-isochroman-1-one HCl (F147) (FL46)

2,N-Dimethyl-benzamide (1 g, 6.7 mmol) and 4′-chloro-(4-methyl-piperazin-1-yl)-propiophenone yielded 20 mg (2%) of the title compound. Mp>250° C. (decomp.). ¹H NMR (500 MHz) δ2.12-2.61 (m, 15H), 3.46-3.56 (m, 2H), 7.19-7.32 (m, 6H), 7.48 (t, 1H, J=6.5 Hz), 7.99 (d, 1H, J=6.5 Hz), 12.40 (s, 1H). ¹³C NMR (125 MHz) δ26.2, 37.9, 39.3, 45.8, 46.6, 52.5, 53.1, 54.9, 84.9, 126.5, 127.4 (2 C:s), 128.3 (2 C:s), 128.6, 128.7, 131.3, 132.4, 133.2, 138.4, 144.7, 170.5. HRTofMS calcd for C₂₂H₂₆ClN₂O₂ (M+H) m/z 385.1683, found 385.1694.

3-(2-Dimethylaminoethyl)-3-(4-trifluoromethyl-phenyl)-isochroman-1-one HCl (F179) (FL47)

2,N-Dimethyl-benzamide (1 g, 6.7 mmol) and 2-dimethylamino-4′-trifluoromethyl-propiophenone yielded 60 mg (6.3%) of the title compound. Mp>250° C. (decomp.). ¹H NMR (500 MHz) δ2.69-2.86 (m, 9H), 3.21-3.28 (m, 1H), 3.48-3.58 (m, 2H), 7.16 (d, 1H, J=7.0 Hz), 7.31 (t, 1H, J=7.0 Hz), 7.46-7.58 (m, 5H), 7.98 (d, 1H, J=7.2 Hz), 12.45 (bs, 1H). ¹³C NMR (125 MHz) δ36.2, 38.8, 42.6, 43.7, 53.2, 83.6, 123.5 (q, ¹J_{CF}=270.0 Hz), 124.1 (2 C:s), 125.4, 126.1, 128.1 (2 C:s, q, ³J_{CF}=5.0 Hz), 130.5 (q,²J_{CF}=32.6 Hz), 134.7, 136.1 (2 C:s), 144.4, 163.9. Anal. Calcd for C₂₀H₂₁ClF₃NO₂: C, 60.1; H, 5.3; N, 3.5. Found: C, 60.5; H, 5.6; N, 3.6.

3-(4-Chlorophenyl)-3-(2-dimethylaminoethyl)-7-methyl-isochroman-1-one HCl (F190) (FL49)

2,5,N-Trimethyl-benzamide (1 g, 6.1 mmol) and 4′-chloro-2-dimethylamino-propiophenone yielded 150 mg (13%) of the title compound. Mp>250° C. (decomp.). ¹H NMR (400 MHz) δ2.33 (s, 3H), 2.63-2.84 (m, 9H), 3.17-3.21 (m, 1H), 3.38-3.49 (m, 2H), 7.05 (d, 1H, J=7.8 Hz), 7.29-7.32 (m, 5H), 7.82 (d, 1 H, J=2.0 Hz), 12.70 (bs, 1H). ¹³C NMR (100 MHz) δ21.0, 36.3, 38.8, 42.5, 43.9, 53.3, 83.7, 123.9, 126.3 (2 C:s), 127.8, 129.3 (2 C:s), 130.3, 133.3, 134.3, 135.6, 138.1, 138.5, 164.4. Anal. Calcd for C₂₀H₂₃Cl₂NO₂: C, 63.2; H, 6.1; N, 3.7. Found: C, 63.1; H, 6.2; N, 3.9.

3-(4-Chlorophenyl)-3-(2-dimethylaminoethyl)-5-methyl-isochroman-1-one HCl (F194) (FL50)

2,3,N-Trimethyl-benzamide (1 g, 6.1 mmol) and 2-dimethylamino-4′-chloro-propiophenone yielded 260 mg (23%) of the title compound. Mp 243.6-244.3° C. (decomp.). ¹H NMR (400 MHz) δ2.28 (s, 3H), 2.65-2.90 (m, 9H), 3.18-3.22 (m, 1H), 3.32 (d, 1H, ²J=16.5 Hz), 3.48 (d, 1H, ²J=16.5 Hz), 7.21-7.37 (m, 6H), (d, 1H, 6.5 Hz), 12.70 (bs, 1H). ¹³C NMR (100 MHz) δ19.2, 36.1, 36.6, 42.8, 44.3, 53.4, 81.3, 124.5 (2 C:s), 126.4, 128.0 (2 C:s), 128.2, 129.6, 134.6, 135.1, 135.8, 136.3, 138.9, 164.8. Anal. Calcd for C₂₀H₂₃Cl₂NO₂: C, 63.2; H, 6.1; N, 3.7. Found: C, 63.0; H, 6.3; N, 4.0.

3-(2-Dimethylaminoethyl)-3-(4-methyl-phenyl)-isochroman-1-one HCl (F197) (FL51)

2,N-Dimethyl-benzamide (1 g, 6.7 mmol) and 2-dimethylamino-4′-methyl-propiophenone yielded 150 mg (17%) of the title compound. Mp>250° C. (decomp.). ¹H NMR (400 MHz) δ2.25 (s, 3H), 2.64-2.76 (m, 9H), 3.15-3.21 (m, 1H), 3.47-3.48 (m, 2H), 7.01 (d, 2H, J=8.1 Hz), 7.14 (d, 1H, J=7.3 Hz), 7.19-7.31 (m, 3H), 7.43-7.47 (m, 1H), 7.99 (d, 1H, J=7.7 Hz) 12.80 (bs, 1H). ¹³C NMR (100 MHz) δ20.1, 36.5, 39.3, 42.6, 43.9, 53.7, 84.1, 124.5, 124.8 (2 C:s), 128.0 (2 C:s), 129.9 (2 C:s), 130.1, 134.6, 136.7, 136.8, 138.3, 164.5. Anal. Calcd for C₂₀H₂₄ClNO₂: C, 69.5; H, 7.0; N, 4.1. Found: C, 69.4; H, 7.0; N, 4.3.

3-(2-Dimethylaminoethyl)-3-(4-methoxy-phenyl)-isochroman-1-one HCl (F198) (FL52)

2,N-Dimethyl-benzamide (1 g, 6.7 mmol) and 2-dimethylamino-4′-methoxy-propiophenone yielded 50 mg (6.3%) of the title compound. Mp>250° C. (decomp.). ¹H NMR (500 MHz) δ2.62-2.80 (m, 9H), 3.16-3.22 (m, 1H), 3.44-3.52 (m, 2H), 3.76 (s, 3H), 6.83 (d, 2H, J=8.2 Hz), 7.17 (d, 1H, J=7.0 Hz), 7.25-7.29 (m, 2H), 7.32 (m, 1H), 7.48 (m, 1H), 8.01 (d, 1H, J=8.1 Hz), 12.85 (bs, 1H). ¹³C NMR (125 MHz) δ36.8, 39.6, 42.7, 44.2, 53.7, 55.4, 84.1, 114.7, 124.7, 126.3 (2 C:s), 127.8, 128.1 (2 C:s), 130.2, 131.7, 134.7, 136.9, 159.5, 164.7.

3-(4-Chlorophenyl)-3-dimethylaminomethyl-isochroman-1-one HCl (F106) (FL18)

2,N-Dimethyl-benzamide (1 g, 6.1 mmol) and 4-chloro-2-(3-dimethylamino)-propiophenone yielded 175 mg (16%) of the title compd. Mp>250° C. (decomp.). ¹H NMR (500 MHz) δ2.88 (s, 3H), 3.13 (s, 3H), 3.50 (d, 1H, J=14.5 Hz), 3.74 (d, 11H, J=14.5 Hz), 3.94 (d, 11H, J=16.5 Hz), 4.30 (d, 1H, J=16.5 Hz), 7.24-7.29 (m, 4H), 7.42-7.48 (m, 3H), 7.91 (d, 1H, J=7.0 Hz), 12.4 (s, 1H). ¹³C NMR (125 MHz) δ35.1, 44.5, 46.7, 65.2, 83.2, 124.0, 127.1 (2 C:s), 128.0, 128.2, 129.5 (2 C:s), 130.1, 134.8, 135.2, 136.4, 137.1, 163.2. Anal. Calcd for C₁₈H₂₀ Cl₂NO₂: C, 61.4; H, 5.5; N, 4.0. Found: C, 61.5; H, 5.5; N, 3.9.

3-(2-Dimethylaminoethyl)-3-(3-methoxyphenyl)-isochroman-1-one HCl (F210)

2,N-Dimethyl-benzamide (1.6 g, 10.6 mmol) and 3-dimethylamino-1-(3-methoxyphenyl)-propanone yielded 40 mg (2.2%) of the title compd. Mp>250° C. (decomp.). ¹H NMR (400 MHz) δ2.66-2.82 (m, 9H), 3.13-3.23 (m, 1H), 3.44-3.52 (m, 2H), 3.76 (s, 3H), 6.74 (dd, 1H, J=2.2, 8.4 Hz), 6.86 (s, 1H), 6.89 (d, 1H, J=7.7 Hz), 7.15-7.32 (m, 3H), 7.45-7.48 (m, 1H), 8.00 (d, 1H, J=8.8 Hz), 12.75 (bs, 1H). ¹³C NMR (100 MHz) δ36.3, 39.2, 42.4, 44.1, 53.3, 55.2, 83.8, 111.0, 113.5, 117.1, 124.5, 128.0, 128.1, 130.1, 130.4, 134.7, 136.7, 141.4, 160.1, 164.5.

3-(2-Dimethylaminoethyl)-3-(3-fluorophenyl)-isochroman-1-one HCl (F232)

2,N-Dimethyl-benzamide (0.3 g, 2.0 mmol) and 3-dimethylamino-1-(3-fluorophenyl)-propanone yielded 60 mg (17%) of the title compd. Mp>250° C. (decomp.). ¹H NMR (400 MHz) δ2.60-2.84 (m, 9H), 3.16-3.22 (m, 1H), 3.44-3.53 (m, 2H), 6.90-7.47 (m, 7H), 7.97 (d, 1H, J=8.7 Hz), 12.50 (bs, 1H). ¹³C NMR (100 MHz) δ36.4, 42.7, 43.9, 53.3, 83.5, 112.5 (d, ²J_{CF}=36.7 Hz), 115.4 (d, ²J_{CF}=29.8 Hz), 120.6, 124.3, 127.9, 128.1, 130.2, 131.1 (d, ³J_{CF}=9.9 Hz), 134.7, 136.3, 142.7 (d, ³J_{CF}=9.9 Hz), 163.3 (d, ¹J_{CF}=275 Hz), 164.1.

3-(2-Dimethylaminoethyl)-3-(2-methoxyphenyl)-isochroman-1-one HCl (F210)

2,N-Dimethyl-benzamide (0.7 g, 4.4 mmol) and 3-dimethylamino-1-(2-methoxyphenyl)-propanone yielded 50 mg (6.3%) of the title compd. Mp>250° C. (decomp.). ¹H NMR (500 MHz) δ2.52-2.74 (m, 8H), 3.17-3.27 (m, 2H), 3.44-3.47 (m, 2H), 3.96 (s, 3H), 6.81-6.88 (m, 2H), 7.09-7.28 (m, 4H), 7.41-7.44 (m, 1H), 7.98 (d, 1H, J=7.5 Hz), 12.45 (bs, 1H). ¹³C NMR (125 MHz) δ33.2, 36.7, 42.3, 43.9, 53.7, 55.8, 84.2, 111.8, 120.7, 124.4, 126.6, 127.3, 127.6, 127.8, 129.4, 129.9, 134.3, 137.4, 155.1, 164.7.

3-(2-Dimethylaminoethyl)-3-(4-phenoxyphenyl)-isochroman-1-one HCl (F241)

2,N-Dimethyl-benzamide (1.2 g, 4.4 mmol) and 3-dimethylamino-1-(4-phenoxyphenyl)-propanone yielded 650 mg (38%) of the title compd. Mp=245.2-246.3° C. (decomp.). ¹H NMR (400 MHz) δ2.60-2.84 (m, 8H), 3.06-3.25 (m, 2H), 3.43-3.52 (m, 2H), 6.89 (d, 2H, J=5.6 Hz), 6.96 (d, 2H, J=8.1 Hz), 7.11-7.18 (m, 2H), 7.24-7.35 (m, 5H), 7.47-7.51 (m, 1H), 8.00 (d, 1H, J=8.0 Hz), 12.65 (bs, 1H). ¹³C NMR (100 MHz) δ36.6, 39.3, 42.6, 44.1, 53.5, 83.8, 118.4 (2 C:s), 119.7 (2 C:s), 119.9, 124.2, 124.5, 126.4 (2 C:s), 128.1, 129.9 (2 C:s), 130.2, 133.9, 134.6, 136.7, 155.9, 157.7, 164.4.

3-(2-Dimethylaminoethyl)-3-(2-chlorophenyl)-isochroman-1-one HCl (F243)

2,N-Dimethyl-benzamide (1.0 g, 6.6 mmol) and 3-dimethylamino-1-(2-chlorophenyl)-propanone yielded 20 mg (2.1%) of the title compd. Mp>250° C. (decomp.). ¹H NMR (400 MHz) δ2.64-2.84 (m, 9H), 3.16-3.24 (m, 1H), 3.48-3.55 (m, 2H), 7.15 (d, 1H, J=8.0 Hz), 7.21-7.35 (m, 5H), 7.45-7.48 (m, 1H), 8.00 (d, 1H, J=8.1 Hz), 12.70 (bs, 1H). ¹³C NMR (100 MHz) δ36.5, 39.3, 42.5, 43.9, 53.5, 84.0, 124.5, 124.8, 124.9, 127.9, 128.0, 128.4, 129.2, 129.3, 130.1, 134.6, 136.6, 139.8, 164.4.

3-(4-Chlorophenyl)-3-(2-diethylaminoethyl)-isochroman-1-one HCl (FL48)

2,N-Dimethyl-benzamide (2.6 g, 17.5 mmol) and 3-diethylamino-1-(2-chlorophenyl)-propanone yielded 500 mg (16%) of the title compd. Mp>250° C. (decomp.). ¹H NMR (400 MHz) 1.24 (t, 3H, J=7.2 Hz), 1.36 (t, 3H, J=7.6 Hz), 2.66-3.19 (m, 8H), 3.42-3.55 (m, 2H), 7.16 (d, 1H, J=7.6 Hz), 7.29-7.35 (m, 5H), 7.49 (m, 1H), 8.01 (dd, 1H, J=1.2, 8.0 Hz), 12.10 (s, 1H). ¹³C NMR (100 MHz) δ8.1, 8.4, 35.8, 39.3, 45.9, 47.3, 47.4, 84.0, 124.3, 126.5 (2 C:s), 128.0, 128.1, 129.4 (2 C:s), 130.2, 134.4, 134.8, 136.5, 138.6, 164.1.

3-(4-Chlorophenyl)-3-(2-dimethylaminoethyl)-4-methyl-isochroman-1-one HCl (FL61)

2-Ethyl-N-methyl-benzamide (1.0 g, 6.1 mmol) and 3-dimethylamino-1-(2-chlorophenyl)-propanone yielded 80 mg (7%) of the title compd. Mp>250° C. (decomp.). ¹H NMR (400 MHz) δ0.86 (d, 3H, J=7.0 Hz), 2.41-2.53 (m, 7H), 2.55-2.61 (m, 1H), 2.76-2.89 (m, 2H), 3.11-3.14 (m, 1H), 7.30-7.35 (m, 1H), 7.42-7.53 (m, 5H), 7.64-7.66 (m, 1H), 8.12 (d, 1H, J=7.4 Hz). ¹³C NMR (100 MHz) δ19.2, 34.0, 41.5, 42.3, 44.6, 53.7, 86.0, 122.5, 126.5 (2 C:s), 128.2, 128.3, 129.6 (2 C:s), 130.5, 134.4, 135.4, 136.9, 144.1, 163.4.

3-(3-Chlorophenyl)-3-(2-dimethylaminoethyl)-isochroman-1-one HCl (FL62)

2,N-Dimethyl-benzamide (1.2 g, 8.0 mmol) and 3-dimethylamino-1-(3-chlorophenyl)-propanone yielded 160 mg (11%) of the title compd. Mp>250° C. (decomp.). ¹H NMR (400 MHz) δ2.66-2.83 (m, 9H), 3.16-3.23 (m, 1H), 3.44-3.54 (m, 2H), 7.19 (d, 1H, J=7.2 Hz), 7.22-7.38 (m, 5H), 7.51 (m, 1H), 8.02 (d, 1H, J=7.6 Hz), 12.70 (s, 1H). ¹³C NMR (100 MHz) δ36.0, 39.0, 42.2, 43.8, 53.2, 83.5, 122.9, 124.2, 125.1, 127.9, 128.2, 128.8, 130.4, 130.7, 134.8, 135.5, 136.3, 142.2, 164.1.

3-(4-Chlorophenyl)-3-(2-dimethylaminoethyl)-8-methyl-isochroman-1-one HCl (FL63)

2,6,N-Trimethyl-benzamide (1.0 g, 6.1 mmol) and 3-dimethylamino-1-(4-chlorophenyl)-propanone yielded 50 mg (5%) of the title compd. Mp>250° C. (decomp.). ¹H NMR (400 MHz) δ2.59-2.80 (m, 12H), 3.16-3.25 (m, 1H), 3.38-3.51, (m, 2H), 6.95 (d, 1H, J=8.8 Hz), 7.09 (d, 1H, J=8.4 Hz), 7.21-7.47 (m, 5H), 12.38 (s, 1H). ¹³C NMR (100 MHz) δ22.1, 29.4, 36.2, 39.7, 42.6, 43.9, 82.7, 122.7, 125.8, 126.2 (2 C:s), 129.4 (2 C:s), 131.5, 133.5, 134.3, 137.2, 138.4, 143.1, 163.6.

3-(4-Chlorophenyl)-3-(3-dimethylaminopropyl)-isochroman-1-one HCl (FL64)

2, N-Dimethyl-benzamide (1.0 g, 6.1 mmol) and 4-dimethylamino-1-(4-chlorophenyl)-butanone yielded 250 mg (27%) of the title compd. Mp>250° C. (decomp.). ¹H NMR (400 MHz) δ1.76-1.83 (m, 1H), 1.90-1.99 (m, 1H), 2.16-2.34 (m, 2H), 2.62 (d, 3H, J=5.5 Hz), 2.77 (d, 3H, J=6.2 Hz), 2.92-2.99 (m, 2H), 3.42-3.56 (m, 2H), 7.17 (d, 1H, J=8.4 Hz), 7.23-7.36 (m, 5H), 7.44-7.49 (m, 1H), 7.97 (d, 1H, J=9.2 Hz), 12.45 (s, 1H). ¹³C NMR (100 MHz) δ18.7, 37.9, 39.1, 42.2, 43.4, 57.6, 84.9, 124.5, 126.5 (2 C:s), 127.9, 128.0 (2 C:s), 129.9, 130.0, 133.8, 134.5, 137.0, 139.8, 164.8.

3-(2-Dimethylaminoethyl)-3-(1-naphtyl)-isochroman-1-one HCl (F273)

2,N-Dimethyl-benzamide (1.3 g, 8.8 mmol) and 3-dimethylamino-1-(1-naphtyl)-propanone yielded 105 mg (6.3%) of the title compd. Mp>250° C. (decomp.). ¹H NMR (400 MHz) δ2.54-2.98 (m, 8H), 3.13-3.34 (m, 2H), 3.75 (d, 1H, ²J=16.5 Hz), 4.03 (d, 1H, ²J=16.5 Hz), 7.09-7.32 (m, 4H), 7.44-7.52 (m, 3H), 7.61-7.65 (m, 1H), 7.74 (d, 1H, J=8.1 Hz), 7.84 (d, 1H, J=8.4 Hz), 7.96 (d, 1H, J=7.7 Hz), 12.85 (s, 1H). ¹³C NMR (100 MHz) δ35.1, 37.9, 43.0, 43.3, 53.8, 85.7, 124.3, 124.5, 124.7, 125.7, 126.1, 127.2, 128.0, 130.0, 130.1, 130.4, 133.9, 134.0, 134.6, 135.0, 137.2, 137.6, 164.4.

3-(2-Dimethylaminoethyl)-3-(2-naphtyl)-isochroman-1-one HCl (F276)

2,N-Dimethyl-benzamide (1.3 g, 8.8 mmol) and 3-dimethylamino-1-(2-naphtyl)-propanone yielded 400 mg (24%) of the title compd. Mp>250° C. (decomp.). ¹H NMR (400 MHz) δ2.61 (d, 3H, J=4.8 Hz), 2.69-2.78 (m, 5H), 2.89-2.98 (m, 1H), 3.19-3.29 (m, 1H), 3.56 (s, 2H), 7.16 (d, 1H, J=7.7 Hz), 7.26-7.30 (m, 1H), 7.42-7.51 (m, 4H), 7.77-7.85 (m, 4H), 8.02 (d, 1H, J=7.7 Hz), 12.75 (s, 1H). ¹³C NMR (100 MHz) δ36.4, 39.4, 42.7, 44.1, 53.6, 84.0, 122.0, 124.5, 124.6, 126.9, 127.0, 127.7, 128.0, 128.1, 128.4, 129.7, 130.1, 132.7, 132.9, 134.7, 136.5, 137.0, 164.5.

3-(2-Dimethylaminoethyl)-3-(2-thienyl)-isochroman-1-one HCl (F285)

2,N-Dimethyl-benzamide (1.5 g, 9.8 mmol) and 3-dimethylamino-1-(2-thienyl)-propanone yielded 200 mg (12%) of the title compd. Mp>250° C. (decomp.). ¹H NMR (400 MHz) δ2.71-2.82 (m, 8H), 2.89-2.97 (m, 1H), 3.25-3.34 (m, 1H), 3.43-3.59 (m, 2H), 6.86 (dd, 1H, J=4.0, 8.8 Hz), 6.97 (dd, 1H, J=1.0, 4.4 Hz), 7.17-7.22 (m, 2H), 7.31-7.34 (m, 1H), 7.48-7.52 (m, 1H), 8.00 (dd, 1H, J=1.5, 8.0 Hz), 12.70 (s, 1H). ¹³C NMR (100 MHz) δ37.6, 40.3, 42.8, 43.8, 53.4, 82.7, 124.2, 125.4, 125.9, 127.5, 128.1, 128.2, 130.3, 134.6, 136.5, 143.8, 163.7.

References:

1) Clark S D, Nothacker P, Wang Z, Leslie F M, and Civelli O (2000) The urotensin II receptor, GPR-14, is expressed in discrete brain areas. Soc. Nsci. Abstr. In press.

2) Rye D (1997) Contributions of the pedunculopontine region to normal and altered REM sleep. Sleep 20(9):757-788.

3) McCormick, D A (1989) Cholinergic and noradrenergic modulation of thalamocortical processing. Trends in Neurosciences 12(6):215-221.

4) Sarter M, and Bruno J P (1999) Cortical cholinergic inputs mediating arousal, attentional processing and dreaming: differential afferent regulation of the basal forebrain by telencephalic and brainstem afferents. Neuroscience 95(4):933-952.

5) Hoegberg, T.; Ulff, B.; Renyi, A. L. J. Med. Chem. 1981, 24 (12), 1499

6) Carter, R. H.; Garson, M. J.; Hill, R. A.; Staunton, J.; Sunter, D. C. J. Chem. Soc., Perkin Trans. 1 1981, 471

7) Handa, S.; Jones, K.; Newton, C. G. J. Chem. Soc., Perkin Trans. 1 1995, 12, 1623

What is claimed is:

1. A compound of Formula I, or salts or prodrugs thereof, complexed with a human urotensin II receptor 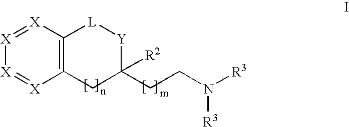
wherein
X is selected from the group consisting of CR¹ and N;
wherein each R¹ is independently and optionally selected from the group consisting of hydrogen, hydroxy, amino, halogen, optionally substituted aryl, optionally substituted heteroaryl, optionally substituted C₃₋₈-cycloalkyl, optionally substituted C₃₋₈ heterocyclyl, optionally substituted C(O)—R, optionally substituted C₁₋₆-alkyl, optionally substituted C₁₋₆-alkoxy, optionally substituted C₂₋₈-alkenyl and optionally substituted C₂₋₈-alkynyl;
R² is selected from the group consisting of optionally substituted aryl and optionally substituted heteroaryl;
each R³ is independently and optionally selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;
L is selected from the group consisting of CRR′, C(O), N(R³), S(O), S(O)₂, O, S, P, and P(O);
Y is absent or selected from the group consisting of CRR′, N—R³, O, S, and P;
m is an integer in the range from 0 to 5, such as 0, 1, 2, 3, 4, or 5;
n is an integer in the range from 0 to 3, such as 0, 1, 2, or 3; and
R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃-8 heterocyclyl.

2. The compound of claim 1, wherein R³ is selected from the group consisting of optionally substituted aryl, optionally substituted C₁₋₆-alkyl, and optionally substituted C₃₋₈-cycloalkyl.

3. The compound of claim 2, wherein said C₁₋₆-alkyl is an optionally substituted C₁₋₆-alkyl(aryl).

4. The compound of claim 1, wherein at most one X is nitrogen, and the remainder are CR¹.

5. The compound of claim 1, wherein R¹ is selected from the group consisting of hydrogen, hydroxy, halogen, C₁₋₆-alkyl, and C₁₋₆-alkoxy.

6. The compound of claim 1, wherein Y is selected from the group consisting of CRR′, N—R³, and oxygen.

7. The compound of claim 1, wherein L is CRR′ or C(O).

8. The compound of claim 1, wherein R² is selected from the group consisting of aryl and heteroaryl, optionally substituted 0 to 3 times.

9. The compound of claim 8, wherein said optional substituents on R² are in the para-position, in the meta-position, or in meta- and para-positions.

10. The compound of claim 8, wherein said optional substituents on R² are selected from the group consisting of optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₁₋₆-alkoxy, optionally substituted C₂₋₈-alkenyl, optionally substituted C₂₋₈-alkynyl, hydrogen, hydroxy, and halogen.

11. The compound of claim 1, wherein L is CRR′ or C(O), Y is oxygen, and X is carbon.

12. The compound of claim 1, wherein n is 1.

13. The compound of claim 1, wherein m is 1.

14. A complex between a compound of claim 1 and a human urotensin II receptor.

15. A compound of Formula II, or a quaternary ammonium salt thereof, 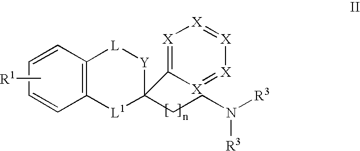
wherein
each of the four R¹ groups is independently selected from the group consisting of hydrogen, hydroxy, halogen, optionally substituted C₁₋₆-alkyl, optionally substituted aryl, optionally substituted heteroaryl, optionally substituted C₃₋₈-cycloalkyl, optionally substituted C₃₋₈ heterocyclyl, optionally substituted C(O)—R optionally substituted C₁₋₆-alkoxy, optionally substituted C₂₋₈-alkenyl and optionally substituted C₂₋₈-alkynyl;
R³ is selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;
X is selected from the group consisting of CR² and N; wherein R² is independently selected from the group consisting of hydrogen, hydroxy, halogen, optionally substituted C₁₋₆-alkyl, optionally substituted aryl, optionally substituted heteroaryl, optionally substituted C₃₋₈-cycloalkyl, optionally substituted C₃₋₈ heterocyclyl, optionally substituted C(O)—R optionally substituted C₁₋₆-alkoxy, optionally substituted C₂₋₈-alkenyl and optionally substituted C₂₋₈-alkynyl;
L and L¹ are independently selected from the group consisting of CRR′, C(O), C(S), N(O), N—R³, S(O), S(O)₂, oxygen, sulfur, phosphorous, and P(O), with the proviso that if L¹ is C(O), L is not CH₂;
Y is selected from the group consisting of CRR′, N—R³, oxygen, sulfur, and phosphorous;
n is an integer in the range from 0 to 5, such as 0, 1, 2, 3; 4 or 5; and
R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃₋₈ heterocyclyl.

16. The compound of claim 15, wherein at most one X is nitrogen, and the remainder are CR².

17. The compound of claim 15, wherein R³ is selected from the group consisting aryl, C₁₋₆-alkyl, and C₃₋₈-cycloalkyl.

18. The compound of claim 15, wherein n is 1.

19. A compound of Formula I 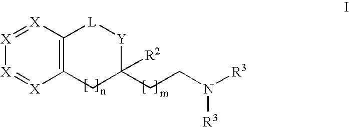
having the same absolute configuration as the compound of Formula III-i, and essentially free from a compound of Formula I having the same absolute configuration as the compound of Formula III-ii 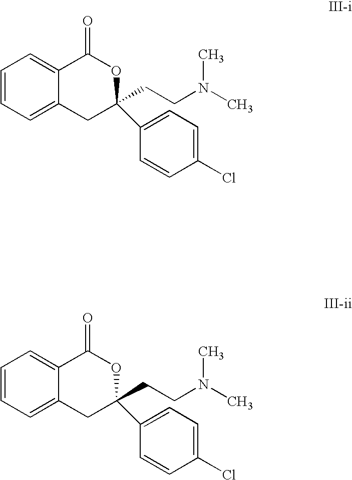
wherein
X is selected from the group consisting of CR¹ and N;
wherein each R¹ is independently and optionally selected from the group consisting of hydrogen, hydroxy, amino, halogen, optionally substituted aryl, optionally substituted heteroaryl, optionally substituted C₃₋₈-cycloalkyl, optionally substituted C₃₋₈ heterocyclyl, optionally substituted C(O)—R optionally substituted C₁₋₆-alkyl, optionally substituted C₁₋₆-alkoxy, optionally substituted C₂₋₈-alkenyl and optionally substituted C₂₋₈-alkynyl;
R² is selected from the group consisting of optionally substituted aryl and optionally substituted heteroaryl;
each R³ is independently and optionally selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;
L is selected from the group consisting of CRR′, C(O), N(R³), S(O), S(O)₂, O, S, P, and P(O);
Y is absent or selected from the group consisting of CRR′, N—R³, O, S, and P;
m is an integer in the range from 0 to 5, such as 0, 1, 2, 3, 4, or 5;
n is an integer in the range from 0 to 3, such as 0, 1, 2, or 3; and
R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃₋₈ heterocyclyl.

20. A compound of Formula I 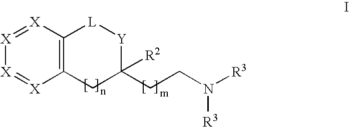
having the same absolute configuration as the compound of Formula III-ii, and essentially free from a compound of Formula I having the same absolute configuration as the compound of Formula III-i 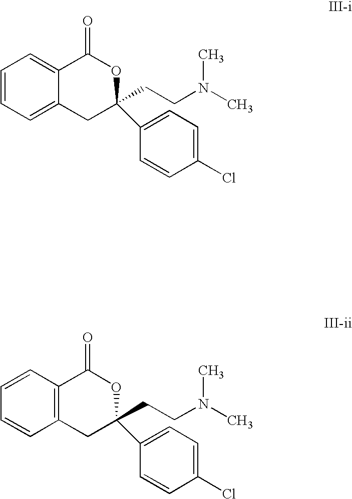
wherein
X is selected from the group consisting of CR¹ and N;
wherein each R¹ is independently and optionally selected from the group consisting of hydrogen, hydroxy, amino, halogen, optionally substituted aryl, optionally substituted heteroaryl, optionally substituted C₃₋₈ -cycloalkyl, optionally substituted C₃₋₈heterocyclyl, optionally substituted C(O)—R optionally substituted C₁₋₆-alkyl, optionally substituted C₁₋₆-alkoxy, optionally substituted C₂₋₈-alkenyl and optionally substituted C₂₋₈-alkynyl;
R² is selected from the group consisting of optionally substituted aryl and optionally substituted heteroaryl;
each R³ is independently and optionally selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;
L is selected from the group consisting of CRR′, C(O), N(R³), S(O), S(O)₂, O, S, P, and P(O);
Y is absent or selected from the group consisting of CRR′, N—R³, O, S, and P;
m is an integer in the range from 0 to 5, such as 0, 1, 2, 3, 4, or 5;
n is an integer in the range from 0 to 3, such as 0, 1, 2, or 3; and
R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃₋₈ heterocyclyl.

21. The compound of claim 15 having the same absolute configuration as the compound of Formula III-i, and essentially free from a compound of claim 15 having the same absolute configuration as the compound of Formula III-ii.

22. The compound of claim 15 having the same absolute configuration as the compound of Formula III-ii, and essentially free from a compound of claim 15 having the same absolute configuration as the compound of Formula III-i.

23. A compound of Formula I with an enantiomeric excess of more than 1% of the 1-R or 1-S enantiomer 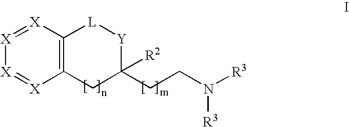
wherein
X is selected from the group consisting of CR¹ and N;
wherein each R¹ is independently and optionally selected from the group consisting of hydrogen, hydroxy, amino, halogen, optionally substituted aryl, optionally substituted heteroaryl, optionally substituted C₃₋₈-cycloalkyl, optionally substituted C₃₋₈ heterocyclyl, optionally substituted C(O)—R optionally substituted C₁₋₆-alkyl, optionally substituted C₁₋₆-alkoxy, optionally substituted C₂₋₈-alkenyl and optionally substituted C₂₋₈-alkynyl;
R² is selected from the group consisting of optionally substituted aryl and optionally substituted heteroaryl;
each R³ is independently and optionally selected from the group consisting of hydrogen, optionally substituted aryl, optionally substituted heteroaryl, optionally substituted C₃₋₈-cycloalkyl, optionally substituted C₃₋₈ heterocyclyl, optionally substituted C₁₋₆-alkyl, optionally substituted C₂₋₈-alkenyl and optionally substituted C₂₋₈-alkynyl, and optionally substituted C(O)—R; or two R³s and the nitrogen atom to which they are attached form a heterocyclyl or heteroaryl ring;
L is selected from the group consisting of CRR′, C(O), N(R³), S(O), S(O)₂, O, S, P, and P(O);
Y is absent or selected from the group consisting of CRR′, N—R³, O, S, and P;
m is an integer in the range from 0 to 5, such as 0, 1, 2, 3, 4, or 5;
n is an integer in the range from 0 to 3, such as 0, 1, 2, or 3; and
R and R′ are independently selected from the group consisting of hydrogen, hydroxy, amino, halogen, optionally substituted aryl, optionally substituted C₁₋₆-alkyl, optionally substituted C₁₋₆-alkoxy, optionally substituted C₂₋₈-alkenyl and optionally substituted C₂₋₈-alkynyl optionally substituted heteroaryl, optionally substituted C₃₋₈-cycloalkyl, and optionally substituted C₃₋₈ heterocyclyl.

24. The compound of claim 15 having an enantiomeric excess of more than 1% of the 1-R or 1-S enantiomer.

25. The compound according to any one of claims 23 to 24, wherein the enantiomeric excess is at least 50%.

26. The compound according to any one of claims 23 to 24, wherein the enantiomeric excess is at least 95%.

27. The compound according to any one of claims 23 to 24, wherein the enantiomeric excess is at least 99%.

28. A compound of Formula III,

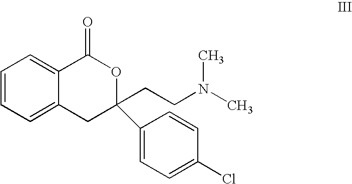

29. A compound selected from the group consisting of 3-(2-Dimethylaminoethyl)-3-phenyl-isochroman-1-one; 3-(2-Dimethylaminoethyl)-5-methyl-3-phenyl-isochroman-1-one; 3-(2-Dimethylaminoethyl)-7-methyl-3-phenyl-isochroman-1-one; 3-(2-Dimethylaminoethyl)-6-methyl-3-phenyl-isochroman-1-one; 3-(2-Dimethyl-aminoethyl)-5-methoxy-3-phenyl-isochroman-1-one; 3-(2-Dimethylaminoethyl)-5-fluoro-3-phenyl-isochroman-1-one; 3-(4-Chlorophenyl)-3-[2-(pyrrolidin-1-yl)-ethyl]-isochroman-1-one; 3-(4-Chlorophenyl)-3-(2-dimethylaminoethyl)-6-methyl-isochroman-1-one; 3-(4-Chlorophenyl)-3-[2-(piperidin-1-yl)-ethyl]-isochroman-1-one; 3-(4-Chloro-phenyl)-3-(2-dimethylaminoethyl)-5-methoxy-isochroman-1-one; 3-(4-Chlorophenyl)-3-[2-(morpholin-1-yl)-ethyl]-isochroman-1-one; 3-(4-Chlorophenyl)-3-[2-(4-methyl-piperazin-1-yl)-ethyl]-isochroman-1-one; 3-(2-Dimethylaminoethyl)-3-(4-trifluoro-methyl-phenyl)-isochroman-1-one; 3-(4-Chlorophenyl)-3-(2-dimethylaminoethyl)-7-methyl-isochroman-1-one; 3-(4-Chlorophenyl)-3-(2-dimethylaminoethyl)-5-methyl-isochroman-1-one; 3-(2-Dimethylaminoethyl)-3-(4-methyl-phenyl)-isochroman-1-one; 3-(2-Dimethylaminoethyl)-3-(4-methoxy-phenyl)-isochroman-1-one; 3-(2-Dimethylamino-ethyl)-3-(3-methoxyphenyl)-isochroman-1-one; 3-(2-Dimethylaminoethyl)-3-(3-fluoro-phenyl)-isochroman-1-one; 3-(2-Dimethylaminoethyl)-3-(2-methoxyphenyl)-isochroman-1-one; 3-(2-Dimethylaminoethyl)-3-(4-phenoxyphenyl)-isochroman-1-one; 3-(2-Dimethylaminoethyl)-3-(2-chlorophenyl)-isochroman-1-one; 3-(4-Chlorophenyl)-3-(2-diethylaminoethyl)-isochroman-1-one; 3-(4-Chlorophenyl)-3-(2-dimethylaminoethyl)-4-methyl-isochroman-1-one; 3-(3-Chlorophenyl)-3-(2-dimethylaminoethyl)-isochroman-1-one; 3-(4-Chlorophenyl)-3-(2-dimethylaminoethyl)-8-methyl-isochroman-1-one; 3-(4-Chlorophenyl)-3-(3-dimethylaminopropyl)-isochroman-1-one; 3-(2-Dimethylamino-ethyl)-3-(1-naphtyl)-isochroman-1-one; 3-(2-Dimethylaminoethyl)-3-(2-naphtyl)-isochroman-1-one; and 3-(2-Dimethylaminoethyl)-3-(2-thienyl)-isochroman-1-one.

30. A pharmaceutical composition comprising a compound of claim 1 or a compound of claim 15, together with a pharmaceutically acceptable excipient, diluent, or carrier.

31. A method of treating diseases and disorders for which activation or modulation of the urotensin II receptor produces a physiologically beneficial response in said disease or disorder comprising administering an effective amount of a compound of any one of claim 1 or 15.

32. The method of claim 31, wherein the diseases and disorders are associated with CNS function.

33. The method of claim 31, wherein said disease is selected from the group consisting of Parkinson's Disease, Alzheimer's Disease, amylotrophic lateral sclerosis, muscular dystrophy, childhood spinal muscular atrophy, progressive spinal muscular atrophy, progressive bulbar palsy, OPCA, ADHD, schizophrenia, insomnia, and Shy Drager syndrome.

34. The method of claim 31, wherein the diseases and disorders are cardiovascular disorders.

35. The method of claim 34, wherein said disease is selected from the group consisting of hypertension; hypotensive states related to shock, sepsis, major surgery and congestive heart failure.

36. A method of altering the vascular pressure in a mammal, comprising identifying a mammal in need thereof and administering an effective amount of a compound of claim 1.

37. A method of altering the heart rate in a mammal, comprising identifying a mammal in need thereof and administering an effective amount of a compound of claim 1.

38. A method of altering the locomotor activity of a mammal, comprising identifying a mammal in need thereof and administering an effective amount of a compound of claim 1.

39. A method of increasing the activity of a urotensin II receptor comprising providing a compound of claim 1 to a system comprising said receptor.

40. A method for augmenting cellular activity in a mammal, comprising identifying a mammal in need thereof and administering an effective amount of a compound of claim 1, thereby activating the signalling of a urotensin II receptor.
